(19) United States
(12) Patent Application Publication
(10) Pub. No.: US 2022/0117461 A1
(43) Pub. Date: Apr. 21, 2022

(54) MANIPULATOR WITH SERIAL ACTUATION

(71) Applicant: W ENDOLUMINAL ROBOTICS LTD., Ariel (IL)

(72) Inventors: Nir SHVALB, Bahan (IL); Oded MEDINA, Ariel (IL)

(21) Appl. No.: 17/442,538

(22) PCT Filed: Mar. 25, 2020
(86) PCT No.: PCT/IL2020/050353

§ 371 Date: Sep. 23, 2021

Related U.S. Application Data
(60) Provisional application No. 62/823,007, filed on Mar. 25, 2019.

(51) Int. Cl. A61B 1/008; A61B 1/00; A61B 1/005; A61B 34/30; A61B 34/00

(52) U.S. Cl. CPC A61B 1/008; A61B 1/00135; A61B 1/00149; A61B 1/0016; A61B 2034/306; A61B 34/30; A61B 34/71; A61B 2034/301; A61B 1/0055

(57) ABSTRACT

A long serial mechanism (LSM) extends from an actuator, distally along a chain of links. Each link comprises a moving mechanism (MM), actuation of which adjusts a position of the link with respect to another of the links. The LSM comprises a shaft, and a series of driving mechanisms (DMs) coupled to the shaft at each link. The LSM has (i) an engaging state in which each DM can potentially transfer rotation of the shaft into actuation of a respective MM, and (ii) a neutral state in which the DMs cannot transfer rotation. Via only (i) transitioning the shaft between the neutral state and the engaging state, and (ii) rotation of the shaft, the actuator is able to cause actuation of the first MM independently from actuation of the second MM, and actuation of the second MM independently of actuation of the first MM. Other embodiments are also described.

### RELATED APPLICATIONS

This application is a PCT Application claiming priority from U.S. Provisional Application No. 62/823,007, filed on 25 Mar. 2019. The contents of all of the above applications are incorporated by reference as if fully set forth herein.

### FIELD AND BACKGROUND OF THE INVENTION

The present invention, in some embodiments thereof, relates to a serial manipulator or serial robot and, more particularly, but not exclusively, to a serial robot with a number of degrees of freedom greater than a number of actuators at a base of the serial robot.

The disclosures of all references mentioned above and throughout the present specification, as well as the disclosures of all references mentioned in those references, are hereby incorporated herein by reference.

### SUMMARY OF THE INVENTION

The present invention, in some embodiments thereof, relates to a serial manipulator or serial robot and, more particularly, but not exclusively, to a serial robot with a number of degrees of freedom greater than a number of actuators at a base of the serial robot.

According to an aspect of some embodiments of the present invention there is provided a serial robot including a base link including one or more actuators, a plurality of links connected from the base link to a distal link, a long serial mechanism (LSM) passing from the base link to the distal link, wherein each link includes a moving mechanism (MM) attached to the LSM, an actuation transfer component, a driving mechanisms DM, and a release mechanism RM.

According to some embodiments of the invention, further including a backlash mechanism (BM).

According to some embodiments of the invention, the LSM is flexible. According to some embodiments of the invention, the LSM is rigid.

According to some embodiments of the invention, a proximal portion of the robot is flexible.

According to some embodiments of the invention, the LSM includes a hollow tube.

According to some embodiments of the invention, the LSM includes an axially-innermost component in the serial robot. According to some embodiments of the invention, the LSM includes an axially outermost component in the serial robot.

According to some embodiments of the invention, the MM includes a protrusion protruding toward the BM and the BM includes a protrusion protruding toward the MM.

According to some embodiments of the invention, the MM includes a cylinder having at least one end shaped at an angle to a plane perpendicular to a longitudinal axis of the cylinder.

According to some embodiments of the invention, the distal link includes two BMs and the MM includes a first protrusion protruding toward a first BM and a second protrusion protruding toward a second BM.

According to some embodiments of the invention, the first protrusion protrudes toward the first BM at a different angle relative to a circumference of the LSM than the second protrusion protrudes toward the second BM.

According to some embodiments of the invention, the first BM includes a protrusion protruding toward the MM at a different angle relative to a circumference of the LSM than a protrusion of the second BM protruding toward the MM.

According to some embodiments of the invention, the BM includes a screw. According to some embodiments of the invention, the BM includes a pin and a slot.

According to some embodiments of the invention, the MM includes a cropped cylinder. According to some embodiments of the invention, the MM includes a screw mechanism. According to some embodiments of the invention, the MM includes a slot and a pin.

According to some embodiments of the invention, the DM includes a leaf spring.

According to some embodiments of the invention, the LSMs can be withdrawn from the serial robot. According to some embodiments of the invention, the LSMs can be withdrawn from the serial robot and subsequently re-inserted into the serial robot. According to some embodiments of the invention, the LSMs can be withdrawn from the serial robot leaving a hollow outer tube.

According to some embodiments of the invention, an apparatus or system including more than one serial robot according to the description herein, arranged to form a closed kinematic loop.

According to some embodiments of the invention, the DM is configured for actuating an effector.

According to an aspect of some embodiments of the present invention there is provided a serial robot including an actuator included in a base of the serial robot, configured to manipulate at least one DOF (degree of freedom), a plurality of links, and at least one long serial mechanism (LSM) designed to pass through each one of the links, designed to transfer actuation of the actuator.

According to some embodiments of the invention, each link includes a moving mechanism (MM) attached to the LSM, an actuation transfer component, a driving mechanisms DM. and a release mechanism RM.

According to some embodiments of the invention, at least some of the links include more than one LSM.

According to some embodiments of the invention, including two long serial mechanisms (LSMs) side-by-side within the links.

According to some embodiments of the invention, the links include a backlash mechanism (BM).

According to some embodiments of the invention, at least one of the links includes at least one effector. According to some embodiments of the invention, at least one of the links includes more than one effector.

According to some embodiments of the invention, the DM is configured for actuating the effector.

According to some embodiments of the invention, the at least one DOF is actuated when engaged by turning the LSM clockwise (CW) or turning counter-clockwise (CCW).

According to some embodiments of the invention, the at least one DOF is actuated when engaged by a linear movement forward or linear movement backward of the LSM.

According to some embodiments of the invention, the at least one DOF is actuated when engaged by a linear movement forward or backward and a rotating movement CW or CCW of the LSM.

According to some embodiments of the invention, a first link includes an actuation transfer component configured so that when the actuator is manipulated in a first direction in a DOF the actuation transfer component causes the link to act upon an adjacent link, and when the actuator is manipulated in a second direction in the DOF the actuation transfer component causes the link to disconnect from acting upon the adjacent link.

According to some embodiments of the invention, the actuation transfer component in at least one link includes a partially cropped screw, enabling the link to transfer actuation to an adjacent link without the link being actuated.

According to some embodiments of the invention, the actuation transfer component in at least one link includes a partially cropped screw.

According to some embodiments of the invention, a first link includes an actuation transfer component configured so that when the actuator is manipulated in a first direction in a DOF the actuation transfer component causes the link to change a direction in space of an adjacent link relative to the first link.

According to some embodiments of the invention, a first link includes an actuation transfer component configured so that when the actuator is manipulated in a first direction in a DOF the actuation transfer component manipulates a first end effector, and when the actuator is manipulated in a second direction the actuation transfer component causes the link to dis-engage from manipulating the first end effector.

According to some embodiments of the invention, a first link further includes an actuation transfer component configured so that when the actuator is manipulated in one direction in a DOF the actuation transfer component manipulates a first end effector, and when the actuator is manipulated in a different direction in the DOF the actuation transfer component manipulates a second, different end effector.

According to some embodiments of the invention, the actuator includes a plurality of actuators.

According to an aspect of some embodiments of the present invention there is provided a method of operating a serial robot including providing a serial robot including an actuator configured to be manipulated in at least one DOF (degree of freedom), a plurality of links, and at least one actuation transfer component included in each one of the links, wherein a first link and a second link have two states, engaged and dis-engaged, and wherein when the first link is engaged with the second link, activating a first link activates the second link.

According to some embodiments of the invention, the engaged state includes two engaged states, a forward engagement (FE) and a backward engagement (BE).

According to some embodiments of the invention, the forward engagement (FE) corresponds to the second link being distal to the first link, and a first actuation transfer component in the first link activates a second actuation transfer component in the second link.

According to some embodiments of the invention, the backward engagement (BE) corresponds to the first link being proximal of the second link, and a second actuation transfer component in the second link activates a first actuation transfer component in the first link.

According to some embodiments of the invention, activating an LSM enough times in one direction results in all moving mechanisms (MMs) related to the LSM being engaged.

According to some embodiments of the invention, activating a first LSM m₁ times in one direction results in engagement of m₁ MMs in a series from a proximal link to a distal link. According to some embodiments of the invention, activating a second LSM m2<m1 times in one direction results in engagement of m2 MMs in a series from the proximal link to the distal link.

According to some embodiments of the invention, the serial robot is in the following state:

a. m2 most proximal MMs are in an engaged configuration,

b. Next one or more MMs are in a configuration where the first LSM is in an engaged state and the second LSM is not in an engaged state,

c. more distal MMs are not engaged.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-1C are simplified illustrations of an example embodiment of the invention;

FIGS. 1D and 1E are simplified illustration of an example embodiment of the invention;

FIGS. 1F-1H are simplified illustrations of an example embodiment of the invention;

FIGS. 1I-1K are simplified illustrations of an example embodiment of the invention;

FIG. 1L is a simplified illustration of an example embodiment of the invention;

FIGS. 1M and 1N show two views of a serial robot according to an example embodiment of the invention;

FIG. 2A is a simplified block diagram of an example embodiment of the invention;

FIG. 2B is a simplified block diagram of an example embodiment of the invention;

FIG. 2C is a simplified block diagram of a portion of an example embodiment of the invention;

FIG. 3 is a simplified line drawing illustration of a serial robot according to an example embodiment of the invention;

FIG. 4 is a simplified line drawing illustration of links of a serial robot according to an example embodiment of the invention;

FIGS. 5A-5D are simplified illustrations of an example embodiment of the invention;

FIG. 5E is a simplified illustration of an example embodiment of the invention;

FIGS. 6A and 6B are simplified line drawing illustrations of two backlash mechanisms according to two example embodiments of the invention;

FIGS. 7A and 7B are simplified line drawing illustrations of two driving mechanisms according to two example embodiments of the invention;

FIGS. 8A and 8B are simplified line drawing illustrations of long serial mechanisms (LSMs) according to an example embodiment of the invention;

FIG. 9 is a simplified line drawing illustration of two long serial mechanisms (LSMs) according to an example embodiment of the invention;

FIGS. 10A and 10B are simplified line drawing illustrations of a link in a serial robot according to an example embodiment of the invention;

FIG. 11 is a simplified line drawing illustration of links of a serial robot according to an example embodiment of the invention;

FIG. 12 is a simplified line drawing illustration showing states and state transitions according to an example embodiment of the invention.

FIG. 13 is a simplified illustration of prior art NOTES approaches for brain surgery;

FIG. 14 is a simplified illustration of NOTES approaches for brain surgery according to an example embodiment of the invention;

FIGS. 15A and 15B are simplified illustrations of ventricles in the brain which an example embodiment of the invention potentially enables to perform NOTES surgery thereon;

FIGS. 16A and 16B are simplified illustrations of prior art fetal interventions; and

FIG. 17 is a simplified illustration of a NOTES cardiac approach according to an example embodiment of the invention.

### DESCRIPTION OF SPECIFIC EMBODIMENTS OF THE INVENTION

The present invention, in some embodiments thereof, relates to a serial manipulator or serial robot and, more particularly, but not exclusively, to a serial robot with a number of degrees of freedom greater than a number of actuators at a base of the serial robot.

In some embodiments of the invention the number of degrees of freedom is very great, a large and even very large multiple of the number of actuators.

Serial robots and serial manipulators are typically designed as a series of links connected by joints that extend from a base to an end-effector.

### Introduction

In the Overview section below general aspects and more specific embodiments of the invention will be described.

In the present Introduction section a first example embodiment is described, in order to demonstrate some general aspects and introduce a reader to the invention. The early introduction of the first example embodiment is intended to facilitate understanding, and not to differentiate the first example embodiment from other example embodiments.

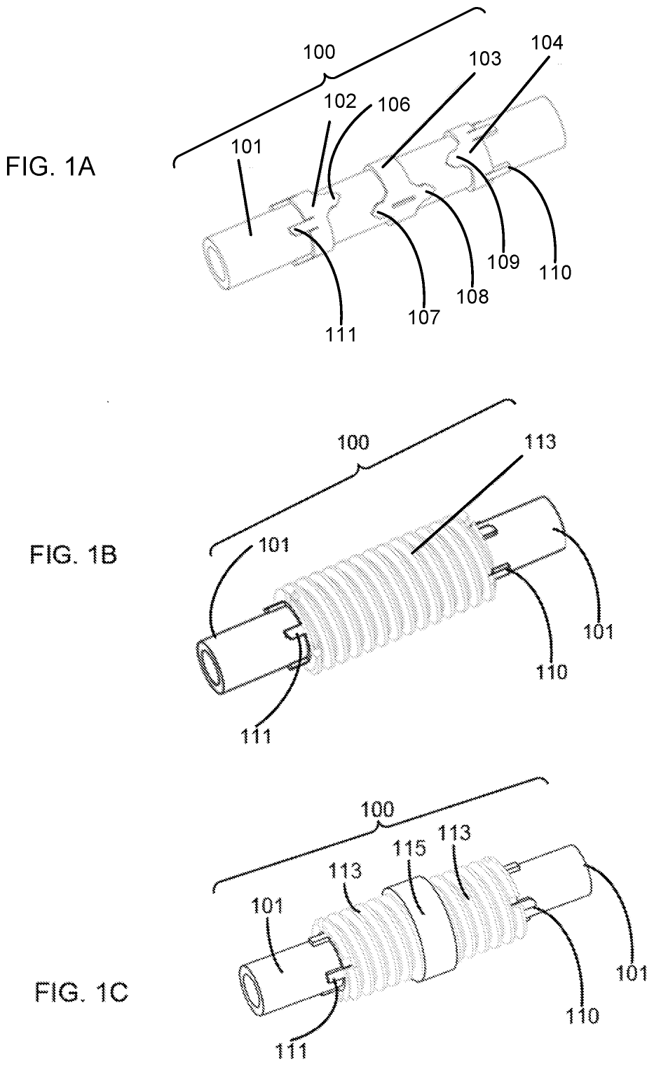

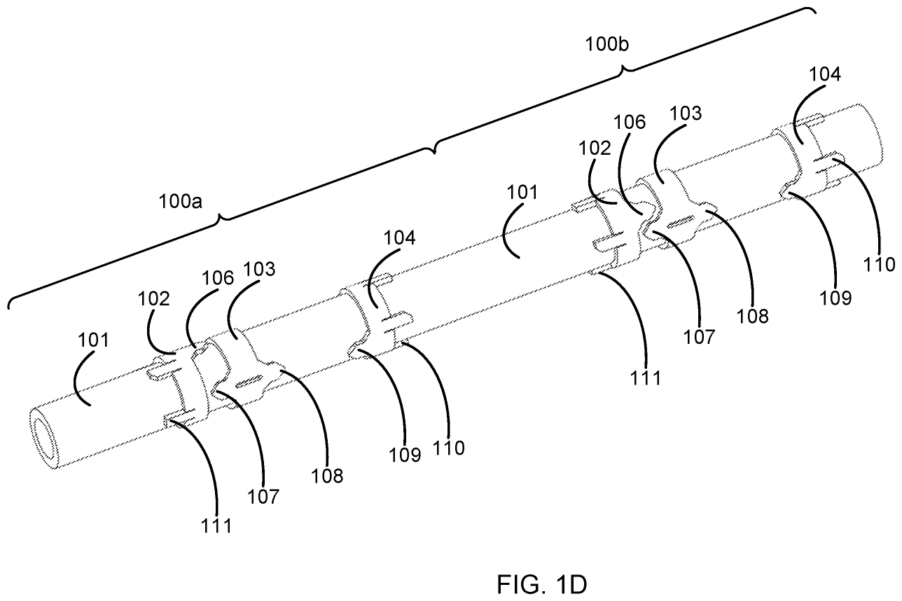

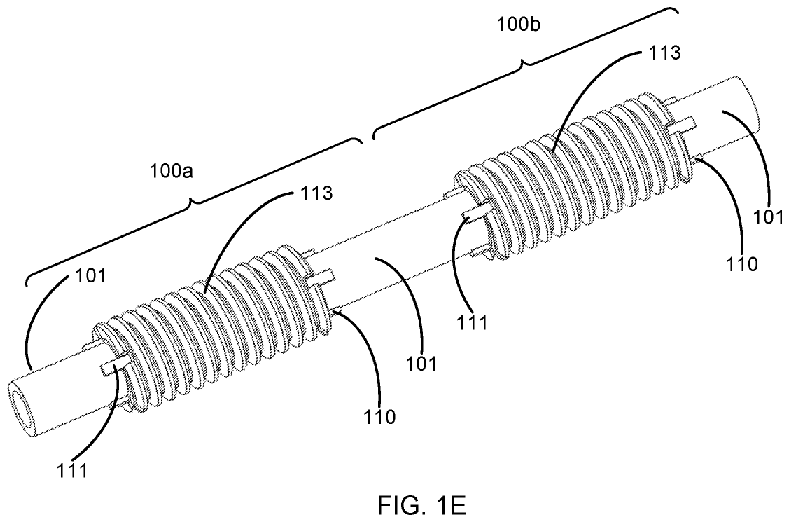

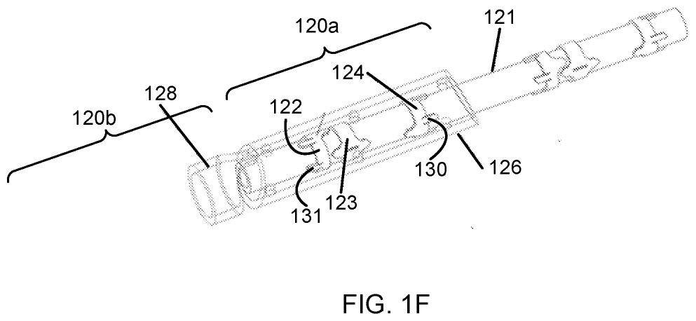

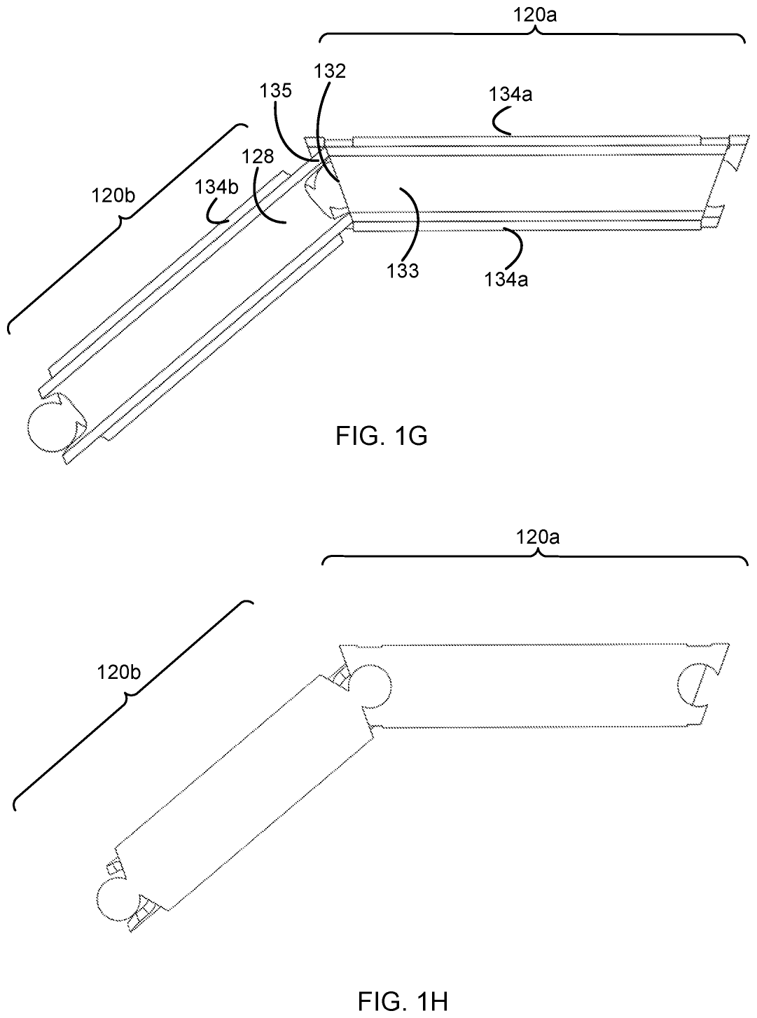

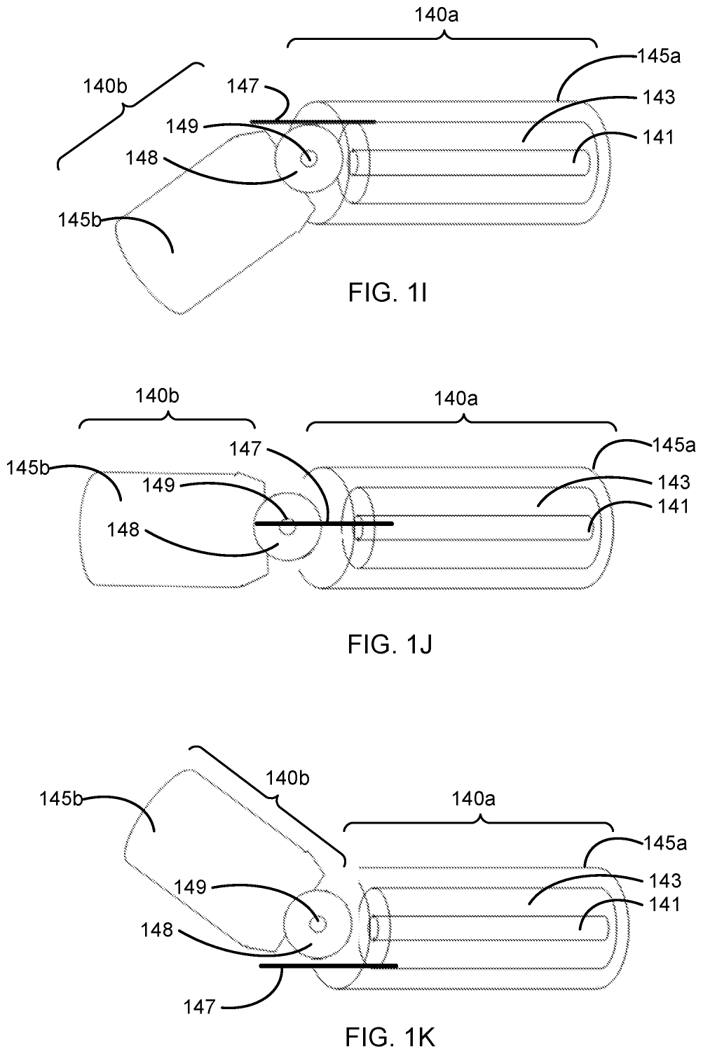

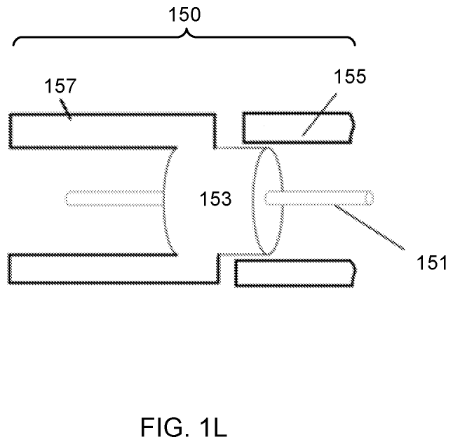

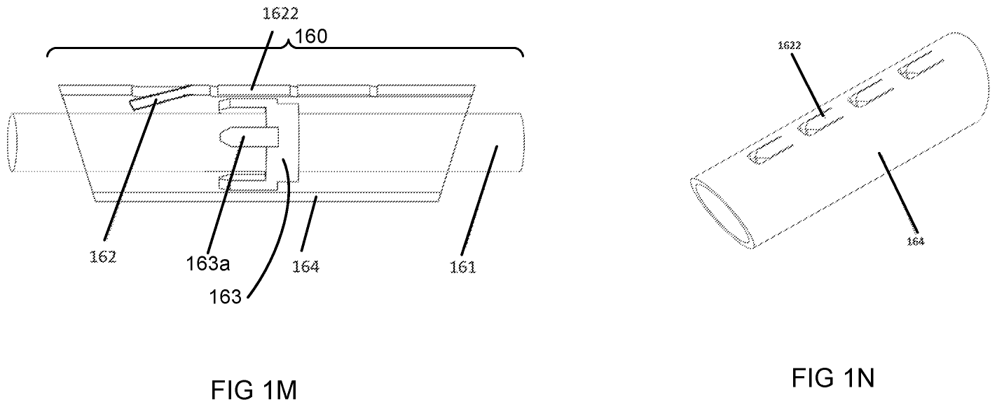

Reference is now made to FIGS. 1A-1E, which are simplified illustrations of an example embodiment of the invention

FIG. 1A shows a section 100 of a serial robot according to an example embodiment of the invention, which is also called a link 100.

The link 100 of the example embodiment includes a shaft 101, on which is fixedly-attached a first band 103. The first band 103 moves with the shaft 101. In some embodiments the shaft 101 may be rigid, and in some embodiments the shaft 101 may be flexible.

In some embodiments, when the shaft 101 is caused to rotate, the first band 103 is caused to rotate. In some embodiments, when the shaft 101 is caused to move forward or backward—the first band 103 moves forward or backward with the shaft 101.

In some embodiments, when the first band 103 is caused to rotate, the shaft 101 is caused to rotate. In some embodiments, when the first band 103 is caused to move forward or backward—the shaft 101 moves forward or backward with the first band 103.

In some embodiments, the shaft 101 has a second band 102 non-fixedly positioned around the shaft 101, with a protrusion 106 jutting toward the first band 103.

In some embodiments the shaft 101 also has a third band 104 non-fixedly positioned around the shaft 101, with a protrusion 109 jutting toward the first band 103.

In some embodiments the first band 103 has a protrusion 107 jutting toward the protrusion 106 of the second band 102.

The shaft 101 may optionally be moved in a first direction, for example backward, and the first band 103 may optionally engage the second band 102 via the protrusion 107 and the protrusion 106. When the first band 103 optionally engages the second band 102, the first band 103 can potentially transfer rotation to the second band 102, and/or the second band 102 can potentially transfer rotation to the first band 103.

In some embodiments the first band 103 has a protrusion 108 jutting toward the protrusion 109 of the third band 104.

The shaft 101 may optionally be moved in a first direction, for example forward, and the first band 103 may optionally engage the third band 104 via the protrusion 108 and the protrusion 109. When the first band 103 optionally engages the third band 104, the first band 103 can potentially transfer rotation to the third band 104, and/or the third band 104 can potentially transfer rotation to the first band 103.

In some example embodiments the first band 103 acts as a driving mechanism (DM). The protrusion 106 of the second band 102 is at a different rotational angle than the protrusion 109 of the third band 104. The different rotational angle enables the first band 103 to engage the second band 102 or the third band 104 at different angles of a rotation, by engaging their relative protrusions.

In various embodiments, a DM optionally serves as a driver which optionally provides motion from one part of the serial robot to another part of the serial robot.

In some example embodiments the first band 103 can engage the second band 102 or the third band 104 for a specific rotational angle, and optionally pull back from engaging, and “skip over” a protrusion by rotating the width of the protrusion.

In some example embodiments the relative angles of the protrusions 107 108 of the first band 103 configure a specific backlash mechanism (BM) configuration.

In various embodiments, a BM optionally serves as a backlash actuator which optionally transfers motion from one part of the serial robot to another part of the serial robot.

In some example embodiments the relative angles of the protrusion 106 of the second band 102 and the protrusion 109 of the third band 104 configure a specific backlash mechanism (BM) configuration.

FIG. 1B shows a screw 113 mounted around the shaft 101 and the first and second bands 102 104.

In some embodiments the screw 113 is fixedly attached to one of the second band 102 or the third band 104. In some embodiments the screw 113 is fixedly attached to both of the second band 102 and the third band 104.

In some embodiments optional rotation-transferring components such as references 110 111 in FIGS. 1A and 1B are used to transfer rotation between the second band 102 and/or the third band 104 to the screw 113.

In some embodiments the shaft 101 acts as a shaft, rotating the first band 103.

In some embodiments the screw 113 acts as a rotator, rotating one or both of the BM(s) 102 104.

The link 100 can optionally be acted upon, by acting upon the shaft 101, to be in one of three states: (state 1) the shaft 101 is engaged with the second band 102; (state 2) the shaft 101 is engaged with the third band 104 (state 3) the shaft 101 is in a neutral position, where rotation of the shaft 101 doesn't rotate the screw 113.

Shifting the link 100 between states is optionally done by a combination of rotations of the shaft 101 and/or moving the shaft 101 forward or backward.

FIG. 1C shows a nut 115 threaded over the screw 113 mounted around the shaft 101 and the first and second bands 102 104 of FIGS. 1A and 1B.

FIG. 1C also shows the optional rotation-transferring components 110 111.

In some embodiments the nut 115 is caused to move forward or backward by a rotation of the screw 113. It is to be appreciated that the screw 113 may or may not be rotated by rotating the shaft 101, depending on the states described above with reference to FIG. 1B. In the embodiment shown in FIG. 1C the nut 115 acts as a Moving Mechanism.

Some mechanisms are now explained. The mechanisms explained may be implemented in embodiments as described in the Figures, and the explanations describe what the various mechanisms can optionally be made to do:

A long serial mechanism (LSM) is a component which passes through one or more link(s), often many links. The LSM may optionally activate the links through which it passes. It is noted that in some embodiments a LSM may be implemented, by way of some non-limiting examples, as a shaft, as an inner tube, and an outer tube.

In various embodiments, an LSM optionally serves as a connector running along a serial robot, from a proximal end or near a proximal end, to a distal end or near a distal end.

A driving mechanism (DM) is a mechanism which provides, or inputs, movement to a link. An LSM may have a DM which inputs movement to more than one link at a time. By way of a non-limiting example, in some embodiments a DM may be a long (flexible or rigid) inner or outer tube which can provide rotation to one or more links in the LSM.

A backlash mechanism (BM) is a mechanism which can be set to a state which optionally transfers, or not, movement from the DM to a moving mechanism (MM) within a link. The movement transferred may be rotational, or linear, or in some embodiments simultaneously rotational and linear.

In various embodiments, a BM optionally serves as an indicator of a state of movement transfer, or no movement transfer, via a link.

The MM is a mechanism which either moves in response to movement input by the DM, or does not move in response to movement input by the DM, based on a setting of the BM.

In various embodiments, a MM optionally serves as a mover, a component which is optionally caused to move by a DM. In some embodiments, the MM is optionally a component which a user is intending to move, and transferring movement to, within one link, or from a distance of one or more links. In some embodiments, the MM may be a tool, for example a surgical tool. In some embodiments, the MM may be connected to a tool or to a surgical tool.

A release mechanism (RM) is a mechanism which optionally causes a LSM to optionally dis-engage from the DM. The release mechanism may also be termed a clutch.

In various embodiments, a RM optionally serves as a clutch which optionally disengages a DM of a link.

By way of a non-limiting example, the mechanisms described above are now used to describe the example embodiment of FIGS. 1A-1C.

In the example embodiment, the link 100 is optionally a link in a LSM; the shaft 101 acts as a driving mechanism (DM); and the first band 103, the second band 104 and the third band 104 act as a backlash mechanism (BM); and the nut 115 acts as a moving mechanism (MM). The DM (shaft 101) may or may not cause movement of the MM (nut 115) based on a state of the BM and on whether the DM is rotated.

Reference is now additionally made to FIGS. 1D and 1E, which are simplified illustration of an example embodiment of the invention.

FIGS. 1D and 1E show two links, a first link 100a and a second link 100b of a LSM, where each one of the links is similar to the link 100 of FIGS. 1A-1B. Similar reference numbers in FIGS. 1D and 1E refer to similar components referenced in FIGS. 1A-1B.

In some embodiments the first band 103 in the first link 100a may optionally be attached to the shaft 101 at a different rotational angle—around the axis of the shaft 101—than the first band 103 in link 100b.

In such embodiments states of the BM of the first link 100a are optionally set at different rotational angles of the shaft 101 than states of the BM of the second link 100b.

In such embodiments movement of the MM of the first link 100a may optionally start at a different time or rotational angle of the shaft 101 than the MM of the second link 100b.

In such embodiments movement of the MM of the first link 100a may operate separately from movement of the MM of the second link 100b, as the BM of the first link 100a may be in a different state than the BM of the second link 100b.

In such embodiments movement of the MM of the first link 100a may operate while the MM of the second link 100b does not move, and vice versa.

In some embodiments the second band 102 in link 100a may optionally be mounted around the shaft 101, or connected to the screw 113, at a different rotational angle around the axis of the shaft 101 than the second band 102 in link 100b. In such embodiments states of the BM of link 100a are optionally set at different rotational angles of the shaft 101.

In such embodiments states of the BM of the first link 100a are optionally set at different rotational angles of the shaft 101 than states of the BM of the second link 100b.

In such embodiments movement of the MM of the first link 100a may optionally start at a different time or rotational angle of the shaft 101 than the MM of the second link 100b.

In such embodiments movement of the MM of the first link 100a may operate separately from movement of the MM of the second link 100b, as the BM of the first link 100a may be in a different state than the BM of the second link 100b.

In such embodiments movement of the MM of the first link 100a may operate while the MM of the second link 100b does not move, and vice versa.

In some embodiments the third band 104 in the first link 100a may optionally be mounted around the shaft 101, or connected to the screw 113, at a different rotational angle around the axis of the shaft 101 than the third band 104 in link 100b. In such embodiments states of the BM of the first link 100a are optionally set at different rotational angles of the shaft 101.

In such embodiments states of the BM of the first link 100a are optionally set at different rotational angles of the shaft 101 than states of the BM of the second link 100b.

In such embodiments movement of the MM of the first link 100a may optionally start at a different time or rotational angle of the shaft 101 than the MM of the second link 100b.

In such embodiments movement of the MM of the first link 100a may operate separately from movement of the MM of the second link 100b, as the BM of the first link 100a may be in a different state than the BM of the second link 100b.

In such embodiments movement of the MM of the first link 100a may operate while the MM of the second link 100b does not move, and vice versa.

By way of a non-limiting example, the mechanisms explained above are now used to describe the states of the example embodiment of FIGS. 1A-1C.

The link 100 can optionally be acted upon, by acting upon the shaft 101, to be in one of three states: (state 1) the BM is engaged, that is, the shaft 101 is engaged with the second band 102; (state 2) the BM is engaged, that is, the shaft 101 is engaged with the third band 104 (state 3) the BM is not engaged, that is, the shaft 101 is in a neutral position, where rotation of the shaft 101 doesn't rotate the screw 113.

The shaft 101 can be used to rotate just the first link 100A, just the second link 100B, or both of the links 100A 100B. A description below of an example of how this is performed explains how by acting upon the shaft 101 more than just two links can be engaged and rotated, and how to select which links are engaged.

The first link 100a optionally has, by way of a non-limiting example:

the protrusion 107 of the first band 103 set at a first angle θ₁₁, relative to some zero angle of rotation of the shaft 101;

the protrusion 108 of the first band 103 optionally set at a second angle θ₁₂;

the protrusion 106 of the BM 102 optionally set at a third angle θ₁₃; and

the protrusion 109 of the BM 104 optionally set at a fourth angle θ₁₄.

The angles θ₁₁ θ₁₂ θ₁₃ θ₁₄ may all be equal, all be different, or some angles equal and some angles different.

The second link 100B optionally has, by way of a non-limiting example:

the protrusion 107 of the first band 103 set at a first angle θ₂₁, relative to some zero angle of rotation of the shaft 101;

the protrusion 108 of the first band 103 optionally set at a second angle θ₂₂;

the protrusion 106 of the BM 102 optionally set at a third angle θ₂₃; and

the protrusion 109 of the BM 104 optionally set at a fourth angle θ₂₄.

The angles θ₂₁ θ₂₂ θ₂₃ θ₂₄ may all be equal, all be different, or some equal some different.

The shaft 101 is optionally advanced (or pulled back) so that when the shaft 101 is rotated, the protrusion 107 (or 108) of the first band 103 of the first link 100A meets the protrusion 106 of the third band 104 (or the protrusion 109 of the second band 102 of the first link 100A). When the shaft 101 is rotated further, the shaft causes the screw 113 of the first link 100A to rotate.

Eventually, the rotation of the shaft 101 causes the protrusion 108 (or 107) of the first band 103 of the second link 100B to meet the protrusion 109 of the third band 104 (or the protrusion 106 of the second band 102) of the second link 100B.

If it is desired that the screw 113 of the second link 100B also rotate, continue the rotation of the shaft 101, and both the screw 113 of the first link 100A and the screw 113 of the second link 100B rotate.

If it is desired that the screw 113 of the second link 100B not rotate, the shaft 101 may be pulled back (or advanced), so as to disengage the protrusion 108 of the first band 103 (or the protrusion 109 of the first band 103) of the second link 100B to disengage from a corresponding protrusion 109 of the third band 104 (or the corresponding protrusion 106 of the second band 102).

The shaft 101 is then optionally rotated so as to cause the protrusion 108 of the first band 103 (or the protrusion 109 of the first band 103) of the second link 100B to rotate past the corresponding protrusion 109 of the third band 104 (or the corresponding protrusion 106 of the second band 102).

In some embodiments the disengagement of the second link 100B by pulling back the shaft 101 also causes a disengagement of the first link 100A.

The shaft 101 is then optionally advanced (or pulled back) to re-engage the protrusion 109 of the first band 103 (or the protrusion 107 of the first band 103) of the first link 100A, and the shaft 101 may optionally be rotated further so as to continue rotating the screw 113 of the first link 100A while not rotating the screw 113 of the second link 100B.

It is noted that by setting the projections 106 107 108 109 of the first link 100A at specific angles θ₁₁ θ₁₂ θ₁₃ θ₁₄ and the projections 106 107 108 109 of the second link 100B at specific angles θ₂₁ θ₂₂ θ₂₃ θ₂₄, control is provided to set when the first link 100A is engaged and when the second link 100B is engaged.

Engaging and/or rotating the screw 113 of the first link 100A and/or the second link 100B is optionally achieved by moving a single activator—in the example embodiment of FIGS. 1A-1E the activator is the shaft 101.

A person who knows the specific angles θᵢ₁ θᵢ₂ θᵢ₃ θᵢ₄ of a specific link i and/or of all the links in a serial robot, and an angle θ(t) at which the shaft 101 is at any time t, can potentially calculate which link is engaged and which is not, can plan by what manipulation of the shaft 101 any link can be engaged and actuated, and can operate the serial robot to engage and/or actuate any one or more links in the serial robot.

A computer which has data regarding the specific angles θᵢ₁ θᵢ₂ θᵢ₃ θᵢ₄ of a specific link i and/or of all the links in a serial robot, and an angle θ(t) at which the shaft 101 is at any time t, can potentially calculate which link is engaged and which is not, can plan by what manipulation of the shaft 101 any link can be engaged and actuated and can operate the serial robot to engage and/or actuate any one or more links in the serial robot.

In some embodiments a computer as described above includes a serial robot control interface which includes lower level commands such as “rotate-clockwise” “rotate-clockwise-by-angle-N” “rotate-counter-clockwise” “rotate-counter-clockwise-by-angle-N” “move-forward” “move-forward-by-M-mm” “move backward” “move backward-by-M-mm” and so on.

In some embodiments a computer as described above includes a serial robot control interface which includes higher level commands such as “engage-link-i”, “disengage-link-i”, “rotate-link-i-clockwise”, “rotate-link-i-counter-clockwise”, “actuate-link-i”, “operate-effector-at-link-i” and so on.

The example of FIGS. 1A-1E, 1M, 1N demonstrates how to engage and/or move and/or rotate a specific number of links by using a lower number of activators.

The example of FIGS. 1A-1E, 1M, 1N demonstrates how to rotate and/or move a specific number of DOFs by using a lower number of activators.

The example of FIGS. 1A-1E, 1M, 1N demonstrates how to cause an effector of a specific link to be activated.

The example of FIGS. 1A-1E, 1M, 1N demonstrates how to select which link is engaged and which link is not engaged.

Reference is now additionally made to FIGS. 1F and 1G, which are simplified illustrations of an example embodiment of the invention.

FIGS. 1F-1H show two sections 120a 120b of a serial robot according to an example embodiment of the invention, which are called links 120a 120b.

The link 120a of the example embodiment includes:

a shaft 121, on which is fixedly-attached a first band 123;

one or more bands 122 124 are non-fixedly positioned around the shaft 121;

one or more protrusions 130 131 protrude from the bands 122 124, as described above with reference to the description of FIGS. 1A-1E;

a joint 128 connects a first link 120a and a second link 120b. The joint 128 may optionally be a universal joint 128;

a shaped cylinder 133 surrounds the first band 123 and is optionally fixedly attached to the one or more bands 122 124; and

an optional sheath 134a surrounds the shaped cylinder 133.

As described above with reference to FIGS. 1A-1E, the first band 123 (which acts in this embodiment as part of the BM) moves with the shaft 121 (which acts in this embodiment as part of the LSM), and by engaging the first band 123 with the one or more one or more bands 122 124 (which act in this embodiment as part of the BM), the one or more bands 122 124 may be rotated by rotating the first band 123.

The shaped cylinder 133 rotates, acting in this embodiment as a MM, with the one or more bands 122 124, when the one or more bands 122 124 are rotated.

In some embodiments a shape of the shaped cylinder 133 interacts with the joint 128.

By way of a non-limiting example, the shaped cylinder 133 has a slanted end 132, on which rests an end 135 of the joint 128.

By way of a non-limiting example the end 132 may include any shape of cut: linear or some other cut.

When the shaped cylinder 133 is optionally rotated between certain angles, as can be understood by a person skilled in the art, the slanted end 132 may optionally push the end 135 of the joint 128, which acts as a driving mechanism (DM), and cause the second link 120b to bend, producing an angle between the first link 120a and the second link 120b.

When the shaped cylinder 133 is optionally rotated between other angles, as can be understood by a person skilled in the art, the slanted end 132 may optionally allow the end 135 of the joint 128 to move toward the first link 120a. The second link 120b optionally aligns or straightens along an axial direction of the first link 120a. In some embodiments a spring or tendon (not shown) pulls the second link 120b to align along the axial direction of the first link 120a

In some embodiments the shaped cylinder 133 may be designed in a way that will enable rotation of an axis of the second link 120b relative to the axis of the first link 120a in two directions which are perpendicular to the axial direction of the first axis 120a, for example constituting a universal joint. FIGS. 1F and 1G show a universal joint 128.

Reference is now made to FIGS. 1I-1K, which are simplified illustrations of an example embodiment of the invention.

FIGS. 1I-1K show a first link 140a and a second link 140b, a shaft 141, a backlash mechanism (BM) 143, optional sheaths 145a 145b of the first link 140a and the second link 140b respectively, a rotation-transferring component 147 and a rotational joint 148 connecting the first link 140a and the second link 140b.

FIGS. 1I-1K show an embodiment in which a shaft 141 enables rotating an axial direction of the second link 140b relative to an axial direction of the first link 140a.

The example embodiment of FIGS. 1I-1K uses the shaft 141 as a Driving Mechanism (DM), and the BM 143 determines whether a rotation of the shaft 141 is transferred to the BM 143. In FIGS. 1I-1K the rotation-transferring component 147 is constructed, by way of a non-limiting example, as a leaf spring 147. The leaf spring 147 is optionally attached to the BM 143.

By way of a non-limiting example:

By rotating the shaft 141 and engaging the BM 143, the leaf spring 147 is brought to push the sheath 145b so that an axial direction of the second link 140b is at an angle relative to an axial direction of the first link 140a, as shown in FIG. 1I. The leaf spring 147 pushes the second link 140b downward, as is enabled by an axis 149 of the joint 148.

By rotating the shaft 141 further, for example by an additional 90 degrees, or by rotating backward 90 degrees, the leaf spring 147 is brought to a different angle. The leaf spring 147 now pushes the sheath 145b in a direction which the direction of the axis 149 of the joint 148 does not enable rotation. Such a case is shown in FIG. 1J, where an axial direction of the second link 140b is aligned with the axial direction of the first link 140a.

By rotating the shaft 141 further, for example by an additional 90 degrees, or by rotating backward by an addition 90 degrees, the leaf spring 147 is brought to push the sheath 145b so that an axial direction of the second link 140b is at an angle relative to an axial direction of the first link 140a, as shown in FIG. 1K. The leaf spring 147 pushes the second link 140b upward, as is enabled by an axis 149 of the joint 148.

It should be noted that in the embodiments described herein the rotating shaft is an inner tube, nevertheless one may apply a rotating shaft LSM which is external to the mechanism, for example an outer rotating sheath.

Reference is now made to FIGS. 1I-1K, which are simplified illustrations of an example embodiment of the invention.

FIGS. 1I-1K show a first link 140a and a second link 140b, a shaft 141, a backlash mechanism (BM) 143, optional sheaths 145a 145b of the first link 140a and the second link 140b respectively, a rotation-transferring component 147 and a rotational joint 148 connecting the first link 140a and the second link 140b.

Reference is now made to FIG. 1L, which is a simplified illustration of an example embodiment of the invention.

FIG. 1L shows a link 150, a shaft 151, a backlash mechanism (BM) 153, an optional first Moving Mechanism (MM) 157 and an optional second Moving Mechanism (MM) 155.

FIG. 1L shows an embodiment in which the BM 153 is controlled by the shaft 151, for example as described above with reference to the example embodiments of FIGS. 1A-1E.

Controlling the BM 153 enables controlling whether rotating the shaft 151 causes, or does not cause, rotation of the MM 157.

Controlling the BM 153 enables controlling whether rotating the shaft 151 causes, or does not cause, rotation of the MM 155.

Reference is now made to FIGS. 1M and 1N, which show two views of a serial robot according to an example embodiment of the invention.

FIGS. 1M and 1N show a 160 of the serial robot which includes:

a shaft 161, on which is fixedly-attached a first band 163, including one or more band-protrusions 163a; and

a shaped cylinder 164 surrounding the first band 163, including one or more protrusions 162, 1622 protruding from the shaped cylinder 164.

FIG. 1M shows the first band 163 with three protrusions. In some embodiments the first band 163 may have a different number of protrusions in the links, which protrusions make up the BM. In some embodiments the first band 163 may have a different number of protrusions in different links.

FIG. 1N shows an embodiment with a protrusions design which has one protrusion as counted when going around a circumference of the cylinder. In other embodiments the BM may include a different number of protrusions as counted when going around a circumference of the cylinder.

As described above with reference to FIGS. 1A-1E, the first band 163 (which acts in this embodiment as part of a RM) moves with the shaft 161 (which acts in this embodiment as part of an LSM), and by engaging the protrusion 162 (which may act in this embodiment as part of a BM), the one or more protrusions 162 1622 may be rotated by rotating the first band 163.

The shaped cylinder 164 can rotate, acting in this embodiment as a MM. In some embodiments a shape of the shaped cylinder 164 interacts with the joint connecting the link 160 and the adjacent links.

### Overview

An aspect of some embodiments of the invention relates to the field of robotics, and more particularly, in some embodiments, to designs of a serial robot with virtually unlimited degrees of freedom activated with a limited number of actuators, optionally located at or near its base.

Typical serial robots (SRs) are characterized by each link being actuated by a separate actuator. Commonly actuators are positioned in vicinity to the links they activate. In tendon-driven SRs actuators are not necessarily positioned in vicinity to the links they activate, but typically have limited capabilities due to their flexibility, and spring like qualities when the tendons are long.

This limits the number of degrees of freedom (DOF) one may have in a small diameter SR, or increases a diameter of the SR. The more actuators and/or the more links, the greater a diameter of a typical SR, due to a need for accommodating a greater number of actuators. For example, when one desires to have a surgical robot for minimal invasive Natural Orifice Transluminal Endoscopic Surgery (NOTES) purposes both limitations—DOF and diameter—can present problems.

Activating an LSM can be done in a forward direction (AFD) or in a backwards direction (ABD).

Considering two consecutive links which are part of a single LSM, and let MM1 MM2 be their corresponding moving mechanisms. If the configuration of their BMs is optionally arranged so that activating MM1 will also activate MM2, one says that the two links are engaged. Engagement can optionally be in one or both of the two options—forward engagement (FE), or backward engagement (BE).

An aspect of some embodiments of the invention introduces a design for a serial robot which involves mechanical design and a conceptual design which enable practically un-limited DOF (practically one can have 20 DOF and more) via a small diameter robot, for example having a diameter of ˜10 mm.

Serial robots which are required to be of small size are typically built with careful engineering and small motors. One may also design a tendon robot—but such robots are typically limited to 7-8 DOF and are typically limited by diameter.

Minimal invasive surgery is typically done through 3-4 incisions in the torso. A single incision surgery is also available for some operations and limited situations due to the lack of dexterity.

Taking, by way of a non-limiting example, Natural Orifice Transluminal Endoscopic Surgery (NOTES). NOTES is described as follows: “scar-less abdominal operations can be performed with an endoscope passed through a natural orifice (mouth, urethra, anus, etc.) then through an internal incision in the stomach, vagina, bladder or colon, thus avoiding any external incisions or scars.”

Natural orifice surgery has gained support as a promising alternative to laparoscopic surgery for abdominal procedures. This led to the first successful series of clinical applications reported in literature, for transvaginal and trans-gastric NOTES. Currently there is no robot that can access inside a body through a narrow passage while performing tasks that require dexterity. Typical tools currently in use are straight long tools with manipulator tips with up to 3 DOF which limit the possibilities of surgery.

Manual tools are typically fabricated as long rods with a wrist joint and a gripper at the tip. Such are not suitable for deep NOTES surgery.

A Da Vinci robot is fabricated as a long rod with a wrist joint and a gripper at the tip. It is not suitable for deep NOTES surgery.

The i-Snake is a short flexible robotic tool. Micro-motors embedded within segments of the i-Snake enable it to move in 7 degrees of freedom by using miniature gears and pulleys. As the motors are embedded in the robot the number of degrees of freedom is fixed, and its current design sets its length to be 20 cm and diameter 14 mm.

Master is a cable based articulation system. It has 7 degrees-of-freedom graspers and a 22 mm diameter. It is an endoscope to be threaded with two grasper arms at its tip.

Viacath is a flexible cable robot consisting of a steerable overtube that houses a standard endoscope and two instrument channels. The Viacath can exert a force up to 3N.

The AnubiscopeVR and the ENDOSAMURAI™ are similar by size and function to the Viacath.

Flex by Medrobotics system is not used as a robot. Rather it is a flexible tube that can reshape in a unique way but not flex like a robot. At the end of the threading process one needs manual tools to manipulate while the Flex system remains still. Its diameter ranges from 17.5 mm to a maximum of 28 mm at a distal end where surgery takes place.

Additional diagnostic endoscopes that are to be threaded include typical endoscopes and more novel endoscopes like Aer-O-Scope, Endotics, Invendoscope and Neoguide. Their typical diameter is above 15 mm.

There is a need for a manipulator according to embodiments of the invention, that optionally includes one or more of the following attributes:

Possesses multiple degrees of freedom (DOF). Desirably more than 5 at the end-effector and optionally an additional number of DOF required for reaching a region of interest.

Enables operation without necessarily inserting electrical motors within the human body.

Enables repeatable motions (termed ‘repeatability’ in the field) that execute capabilities of a robotic system.

Has little or no restrictions on a length to which the manipulator can be inserted. By way of a non-limiting example, in order to perform a Trans-Oral NOTES in adults, one needs to thread an approximately 30 cm long robot with additional capabilities at an end of the robot, so a total length of 54 cm is reasonable. A range of desired length for Endoscopic Surgery can be 20 to 100 cm. Taking an additional view, a manipulator is optionally built that is significantly longer, optionally having a diameter larger than preferred for transluminal surgery. For example a manipulator is optionally constructed to operate in spaces which are less restrictive than a human body lumen, such as within machines, fluid tanks, on an outside of a space station, and so on.

Can exert large forces/moments at its tip. By way of a non-limiting example, a serial robot having an outer diameter of 5 millimeters can exert a lifting force of 500 grams at its tip, perpendicular to a direction of a longitudinal axis of the tip. By way of a non-limiting example, a serial robot having an outer diameter of 2 millimeters can exert a lifting force of 40 grams at its tip, perpendicular to a direction of a longitudinal axis of the tip. By way of a non-limiting example, a two layered HHS flexible rotating shaft with an outer diameter of 2 mm, a 100 g lifting force can be exerted at a tip of a 200 mm long serial robot having a 5 mm diameter. By way of a non-limiting example, a two layered HHS flexible rotating shaft with an outer diameter of 0.9 mm, a 50 g lifting force can be exerted at a tip of a 100 mm long serial robot having a 2.5 mm diameter.

Is thin enough, for example has a diameter less than 9 mm, to be threaded through a small hole, or a vein, or an artery.

Can be operated as a fully actuated robotic arm (as opposed to endoscopes and robotic tip endoscopes like the Master, Viacath, AnubiscopeVR and ENDOSAMURAI™ systems).

Current catheter-like mechanisms typically offer 2-3 DOF at their tip, and therefore may be thin, even having a diameter of 2 mm. current mechanisms that have more DOFs typically have much larger diameters, starting from 8 mm and up.

An example embodiment of the invention may theoretically have almost un-limited DOFs. In some embodiments, for example such which have one LSM, a serial robot diameter is even less than 5 mm.

In some embodiments a serial robot as described herein possesses a potentially unlimited number of DOFs, each link can optionally be designed to have any desired angle in space relative to an adjacent link, potentially with non-varying accuracy.

In some embodiments a serial robot as described herein optionally enables opposite-manipulation. The term opposite-manipulation is used herein to mean that if the serial robot is inserted into a body north to south, the manipulating if performed from south to north—in an opposite direction. The opposite-manipulation on an ability to select which link is manipulated at which time, enabling to manipulate an effector in a distal link and/or to manipulate an effector in a proximal link, and/or to manipulate an effector in any link in between. Opposite-manipulation is a very desirable feature for NOTES systems and such a quality is of importance and is lacking in current technology.

Example embodiments of the invention are described below as potential implementations, in a section titled “potential implementations”.

An aspect of some embodiments relates to a serial robot with a small number of actuators at a first link at a proximal end of the serial robot, a next link movably attached to the first link, and so on—a chain of links serially attached to each other up to a distal end.

An actuation transfer component at the first link optionally engages or disengages driving mechanisms in a second link, the actuation transfer component of the second link optionally engages or disengages driving mechanisms in a third link, and so on.

A driving mechanism at the first link optionally produces an angle change between the first link and the second link, the driving mechanism of the second link optionally produces an angle change between the second link and the third link, and so on.

At any link along the serial robot, from the first, proximal link up to and including the last, distal link, one or more effector(s) may be included in a link. The effector may optionally be a tool such as a medical tool for surgery, or other tool useful for mounting on a serial robot.

An effector mechanism included in a link optionally controls operation of the one or more effector(s).

By controlling actuators at a first link to engage an example link or several, not necessarily consecutive, links somewhere along the series of links, the actuators can optionally cause the example link to change its direction relative to an adjacent previous link, thus optionally shaping the serial robot in space, or within a patient's body when the serial robot is used within a patient's body.

By controlling the actuators at the first link to engage an example link or several, not necessarily consecutive, links somewhere along the series of links, the actuators can optionally operate one or more effectors included in the example link. In some embodiments the example link is a last, distal link of the serial robot. In some embodiments the example link is a link somewhere along the series of links, not necessarily the last, distal link.

In some embodiments the number of actuators included in the first link may be a small number, for example one, two, three or four actuators.

In some embodiments the one or more actuator(s) included in the first link operate(s) on one link, then cause(s) the one link to engage a next link, optionally operate(s) on the next link, then optionally cause(s) the next link to engage a further-next link, and so on. The actuator(s) optionally operate, in a controllable manner, a joint of the link to form a direction and/or shape in space for the series of links.

In some embodiments the one or more actuator(s) operate(s) one or more effector(s) on one link, then cause(s) the one link to engage a next link, optionally operate(s) one or more effector(s) on the next link, then optionally cause(s) the next link to engage a further-next link, and so on. The actuator(s) optionally operate the effector(s) along the serial robots to achieve a desired effect. In medical circumstances the serial robot is optionally used to perform a medical operation, using the effectors, along the serial robot.

In some embodiments a combination of shape/direction operations and effector operations may optionally be performed.

In some embodiments an actuator of the first link optionally has one degree of freedom for movement—for example a movement forward and backward, or a turn clockwise or counterclockwise.

In some embodiments actuators in the first link optionally transfer two degree of freedom for movement—for example a movement forward and backward and a turn clockwise or counterclockwise.

Engaging and Dis-Engaging Links in a Serial Robot

An aspect of some embodiments relates to engaging and dis-engaging a link in a serial robot from an adjacent link.

In some embodiments a single actuation optionally acts upon a backlash mechanism to cause a link to engage an adjacent link, for example, an adjacent distal link, by moving in a first direction or degree of freedom.

In some embodiments repeating the same action with the same actuator optionally acts to cause the just-engaged link to engage an additional, further adjacent link.

In some embodiments moving an actuator forward (or clockwise) optionally acts upon a link to engage a following adjacent link.

In some embodiments moving the actuator forward (or clockwise) again, optionally acts upon an adjacent link to engage an additional, next following adjacent link.

In some embodiments moving the actuator forward (or clockwise) again, optionally acts upon a more-distal link to engage an additional, yet not adjacent link.

An engagement based upon a forward movement is termed a “forward engagement”.

In some embodiments moving the actuator in a reverse direction, for example backward (or counterclockwise), optionally acts upon the adjacent link to engage the additional, next following adjacent link.

In some embodiments moving the actuator in a reverse direction, for example backward (or counterclockwise), optionally acts upon a more-distal link to engage an additional yet not adjacent link.

An engagement based upon a backward movement is termed a “backward engagement”.

The above paragraphs describe two different sequences to perform a first engagement of two links and a second engagement of two links—the first sequence by repeating a direction of movement of the actuation, and the second sequence by performing a first engagement in a first direction and the second engagement in a second, different direction.

In some embodiments a single actuation optionally acts to cause a link to dis-engage an adjacent link, for example, an adjacent distal link, by moving in a first direction or degree of freedom.

In some embodiments repeating the same action with the same actuator optionally acts to cause the just-disengaged link to dis-engage from an additional, typically more proximal adjacent link.

In some embodiments moving an actuator forward (or clockwise) optionally acts upon a first link to dis-engage from a following adjacent link.

In some embodiments moving the actuator forward (or clockwise) again, optionally acts upon the second link, more proximal and adjacent to the first link, to dis-engage from the first.

In some embodiments moving the actuator in a reverse direction, for example backward (or counterclockwise), optionally acts upon the second link to dis-engage from the first link.

The above two paragraphs describe two different sequences to perform a first dis-engagement of two links and a second dis-engagement of two links—the first sequence by repeating a direction of movement of the actuation of the dis-engagement, and the second sequence by performing a first dis-engagement in a first direction and the second engagement in a second, different direction.

In some embodiments a link may optionally include a release mechanism (RM) at both ends of the link.

By way of a non-limiting example, the configuration depicted in 1A-1E acts together as RM and a BM. As the first band 103 can be in a non-engaged position—bands 102, 103 and the third band 104 optionally act as RMs in the embodiment shown in FIGS. 1A-1E.

Note that there can be a first angular difference between the second band 102 and the third band 104 in the first link 100a and a second angular difference between the second band 102 and the third band 104 in the second link 100b. Forward engaging the first band 103 of the first link 100a with the second band 102 of the first link 100a and rotating will eventually cause the first band 103 of the second link 100b to engage with the second band 102 of the second link 100b. This forms a BM.

Operating an Effector of a Link

An aspect of some embodiments relates to operating an effector included in a link.

In some embodiments the effector is optionally designed as a tool for acting upon an environment of the serial robot. By way of some non-limiting examples the effector may be a clamp, a scalpel, a camera for imaging, and so on.

In some embodiments a single actuator optionally acts to operate an effector in a link somewhere along the serial robot.

By way of some non-limiting examples the actuator may be operated to act upon the effector to change a direction of a distal engaged link relative to an adjacent, more-proximal link.

By way of some non-limiting examples the actuator may be operated to act upon the effector to close an effector clamp.

In some embodiments moving the actuator in a first direction, for example forward (or clockwise) optionally acts to operate the effector in a specific direction and moving the actuator in a different direction, for example backward (or counter clockwise) optionally acts to operate the effector in different direction.

Which Links Get Engaged/Dis-Engaged?

In some embodiments an actuator acts via a series of engaged links, and transfers the action of engaging or dis-engaging along a series of engaged links to a most-distal engaged link.

Which Effector Gets Operated?

In some embodiments an actuator acts via a series of engaged links, and transfers the action of operating an effector along a series of engaged links to a most-distal engaged link.

Actuators, Engagement and Disengagement and Operating Effectors

It is noted that an actuator, optionally included in a first link, can serve to engage/disengage a link anywhere along a serial robot.

In some embodiments a same actuator is optionally used to engage/dis-engage and to operate an effector—when the actuator is moved along one degree of freedom, e.g. along a backward/forward direction, the actuator optionally operates to engage/dis-engage a link from an adjacent link, and when the same actuator is optionally moved along another degree of freedom, e.g. clockwise and counterclockwise, the actuator operates an effector.

In some embodiments a single actuator acts to engage/dis-engage and to operate one or more effectors at one or more links along a serial robot.

In some embodiments two actuators act to engage/dis-engage and to operate one or more effectors at one or more links along the serial robot.

In some embodiments there are two actuators at a proximal link, with one actuator acting to engage/dis-engage links, and another actuator acting to operate one or more effectors at one or more links along the serial robot.

An aspect of some embodiments relates to using a serial robot with a plurality of links movably attached to each other along the serial robot, and at least one actuator for operating effectors included in at least one of the serial links, for a medical operation.

An aspect of some embodiments relates to using a serial robot as a part of a platform such as a two or more parallel robots, with a plurality of links movably attached to each other along the serial robot. For example, one SR may be constructed as a SR according to an embodiment of the invention, and one or more additional SRs may be SRs according to prior art.

### Additional Detail

An aspect of some embodiments relates to a concept of having just two motors that can potentially activate infinitely many serial robots and/or serial robots with arbitrary spatial or planar design.

In some embodiments the serial robot includes one or more series of links, each of which includes: a motor to leadscrew to u-joint links, with an inherent backlash to the leadscrew.

A leadscrew (or lead screw), also known as a power screw or translation screw, is a screw used as a linkage in a machine, to translate turning motion into linear motion.

A universal joint (universal coupling, U-joint, Cardan joint, Spicer or Hardy Spicer joint, or Hooke's joint) is a joint or coupling connecting rigid rods whose axes are inclined to each other, and is commonly used in shafts that transmit rotary motion. It consists of a pair of hinges located close together, often oriented at 90° to each other, connected by a cross shaft.

In some embodiments the serial robot includes one or more series of links, each of which includes a flexible shaft such as a HHS (Helical Hollow Strand) or HFS (Hollow Flexible shaft).

In some embodiments, for each one of the two leadscrews a spur gear is positioned.

In some embodiments a motion of a position of a center of gravity (C.G.) of a spur gear is coupled with a joint movement.

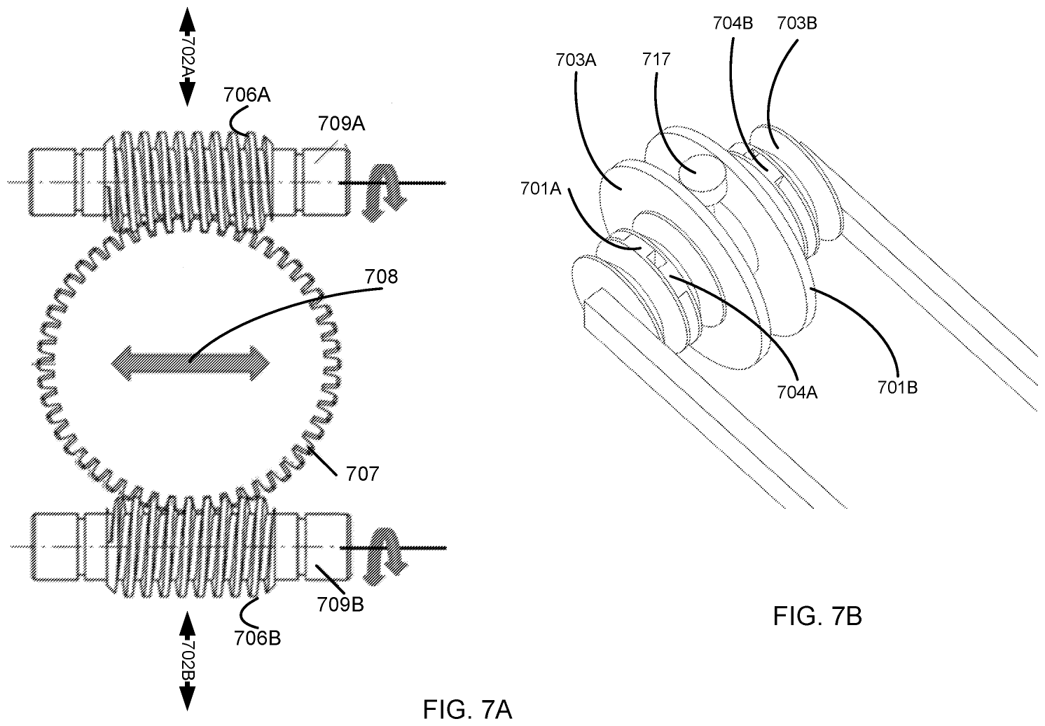

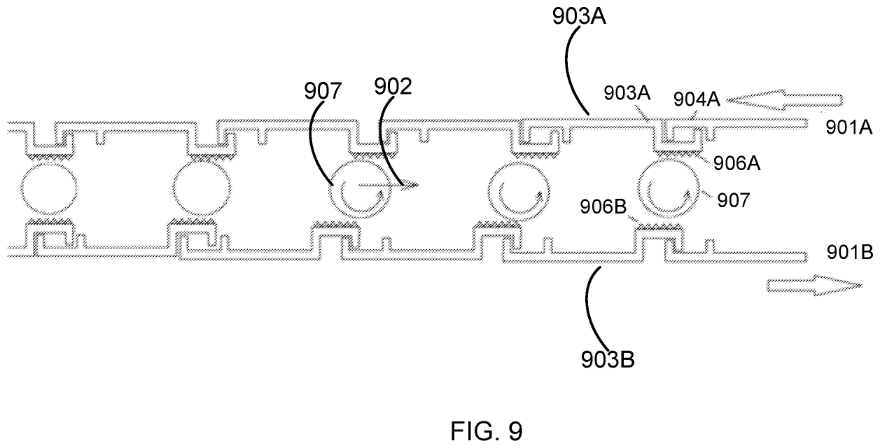

In some embodiments a motion of a position of a C.G. of the spur gear is coupled with a joint movement. In some embodiments the joint movement is not a rotation but a linear movement (see for example FIG. 7A and FIG. 9 and their descriptions).

In some embodiments the spur gear may rotate while its C.G. stays still or moves without rotation or rotates and also moves, optionally when twin leadscrews rotate at a same speed in opposite directions, or rotate at the same speed and same direction, or rotate in different speeds correspondingly (see for example FIG. 7A).

In some embodiments a u-joint is included, and a rotation of a motor has a potential to rotate all lead-screws in a chain of links.

In some embodiments, when there is a backlash in the u-joints, the rotation does not always occur.

In some embodiments, when a leadscrew on a first LSM is rotated at a sufficient angle the leadscrew engages a leadscrew on a second LSM, and the leadscrew of the second LSM starts rotating. A leadscrew can rotate a neighbor leadscrew in a clockwise direction, in a counter clockwise direction, or the first leadscrew may not rotate the neighbor leadscrew.

In some embodiments when a chain of links is rotated in a given sequence of rotations and/or moved in a given sequence of forward-backwards motions, one ends up with a “seemingly” general link-engagement configuration.

In some embodiments the method of operation is optionally simplified. A backlash configuration is optionally designed so that a summation of configurations is enabled, making the backlash configuration space a local vector space. In some embodiments an initial backlash state of each backlash configuration is set to be a same initial backlash state. A design of the mechanism enables a detachment of a serial-chained u-joint mechanism from the leadscrews. Following the detaching serial u-joints are all turned right or left until the mechanism returns to the initial backlash configuration.

In some embodiments, which have two chains of links, a different joint actuation series is designed for a different motor activation chain. The motor activations are optionally summed up. For a given a desired motion a set of previously computed forward-backwards motor activations is optionally calculated.

In some embodiments the serial robot optionally performs as a robot with a potential disadvantage. When one desires >10 DOF the motion of the serial robot may be slow to operate, potentially requiring several operations before engaging an end manipulator. In some embodiments such a situation is optionally mitigated by a precomputation process, optionally optimizing over a path space, for example getting to a location in space fast where the mechanism shape doesn't matter, optionally rather than getting to the location in space vis a configuration space which includes constraints such as, by way of a non-limiting example, avoiding obstacle collisions.

It is to be understood that the invention is not necessarily limited in its application to the details of construction and the arrangement of the components and/or methods set forth in the description herein and/or illustrated in the drawings and/or the Examples. The invention is capable of other embodiments or of being practiced or carried out in various ways.

It is to be understood that the invention is not necessarily limited in its application to the details set forth in the following description or exemplified by the Examples. The invention is capable of other embodiments or of being practiced or carried out in various ways.

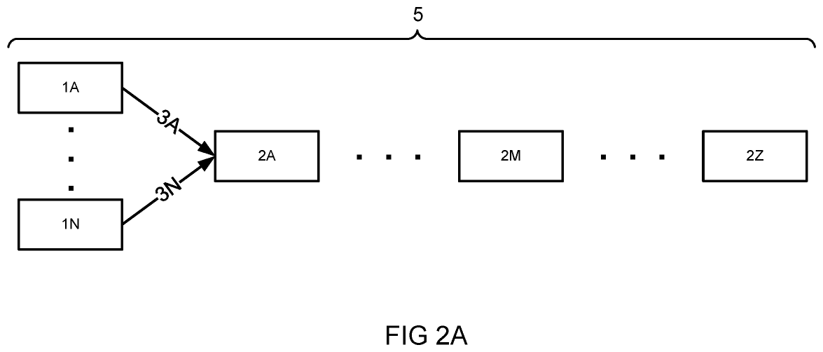

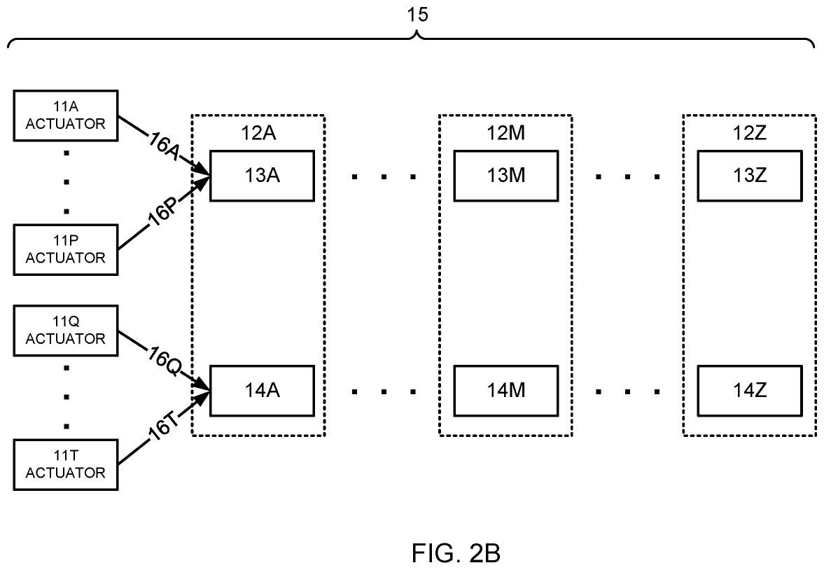

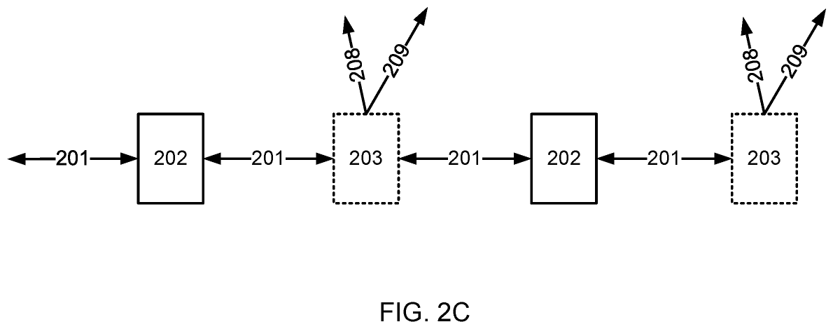

Reference is now made to FIG. 2A, which is a simplified block diagram of an example embodiment of the invention.

FIG. 2A shows a serial robot 5, including one or more actuators 1A . . . 1N configured to manipulate (3A . . . 3N) a first link 2A. The first link 2A is configured to optionally engage with and optionally manipulate an adjacent link, which is configured to optionally engage with, and optionally manipulate a further link, for example link 2M, and so on up to a last link 2Z.

Reference is now additionally made to FIG. 2B, which is a simplified block diagram of an example embodiment of the invention.

FIG. 2B shows a serial robot 15, including one or more actuators 11A . . . 11P configured to manipulate (16A . . . 16P) a first mechanism 13A in a first link 12A, and one or more actuators 11Q . . . 11T configured to manipulate (16Q . . . 16T) a second mechanism 14A in the first link 12A.

The first mechanism 13A in the first link 12A is configured to optionally engage with and optionally manipulate a first mechanism in an adjacent link and the second mechanism 14A in the first link 12A is configured to optionally engage with and optionally manipulate a second mechanism in an adjacent link, which is configured to optionally engage with mechanisms and optionally manipulate mechanism in a further link, for example link 12M, and so on up to a last link 12Z.

Reference is now additionally made to FIG. 2C, which is a simplified block diagram of a portion of an example embodiment of the invention.

FIG. 2C shows a series of links 202 203 of a serial robot according to an example embodiment of the invention. The links 202 203 include mechanisms for engaging with neighbor links, and for transferring manipulation such as by the manipulators 1A . . . 1N and/or 11A . . . 11T of FIGS. 1F and 1G to the neighbor links.

FIG. 2C shows a series of links 202 203 including one or more actuators configured to manipulate 201 a first link which is configured to optionally engage with and optionally manipulate an adjacent link, and so on.

In some embodiments, some links 203 optionally include one or more end effectors 208 209, and some links 202 optionally do not include end effectors 208 209.

In some embodiments, the manipulation 201 of a link 203 optionally acts to manipulate the one or more end effectors 208 209.

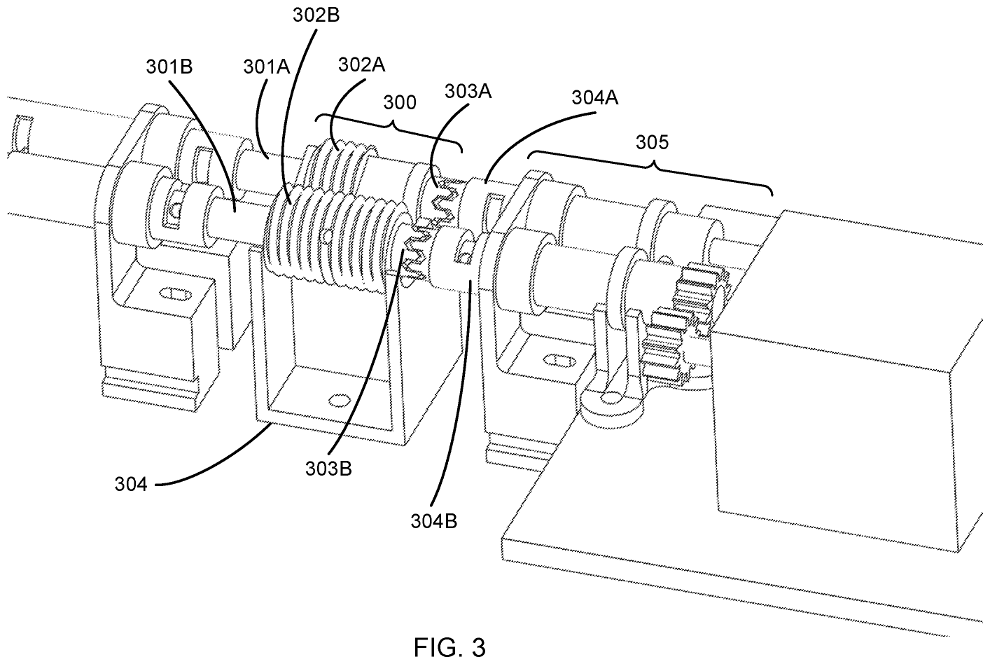

Reference is now additionally made to FIG. 3, which is a simplified illustration of an example embodiment of the invention.

FIG. 3 shows a link 300, with shafts 301A 301B optionally engaged to manipulate screws 302A 302B.

FIG. 3 also shows clutch gears 303A 303B which optionally engage backlash mechanisms 304A 304B.

The shafts 301A 301B, when rotated in a first direction, are optionally engaged to manipulate the screw 302A to move in linear motion. The clutch gears 303A 303B optionally engage the backlash mechanisms 304A 304B with link 300 which optionally receives actuation from a base mechanism 305 which includes the actuators of the serial robot.

The shafts 301A 301B, when rotated in a second direction, are optionally engaged to manipulate the screw 302A, and optionally operate the screw 302A.

In the example embodiment of FIG. 3 as described the screw 302A acts as a driving mechanism of the link 300, the clutch gears 303A 303B act as a Release Mechanism (RM) of the link 300, and the backlash mechanisms 304A 304B, true to their name, acts as backlash mechanisms of the link 300.

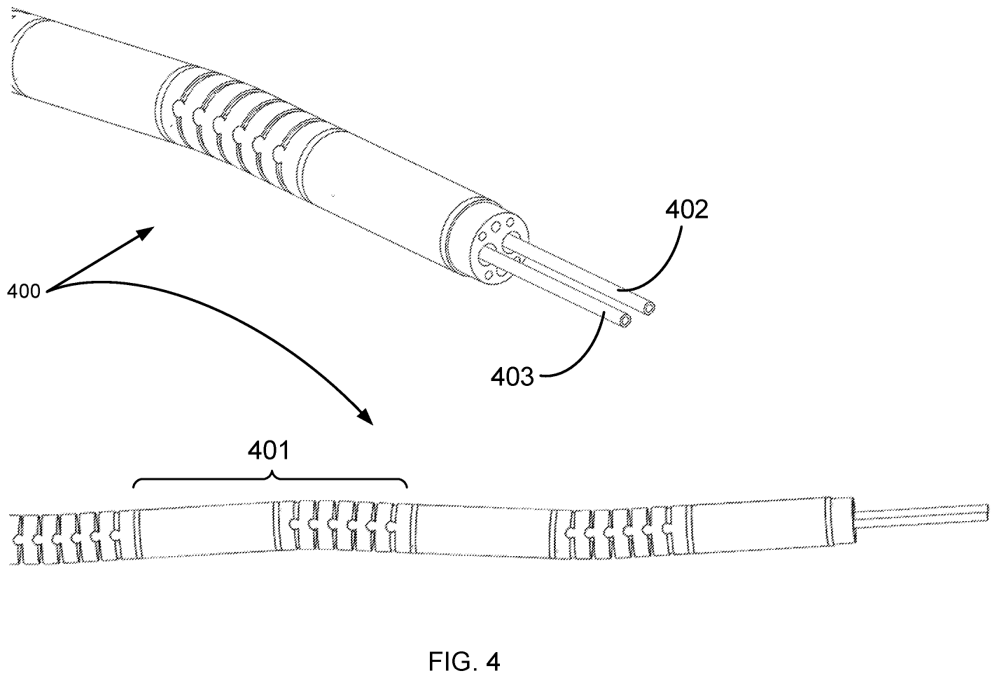

Reference is now additionally made to FIG. 4, which is a simplified illustration of an example embodiment of the invention.

FIG. 4 shows a portion of a serial robot 400, with several links 401, and two actuation transfer components 402 403.

FIG. 4 shows the portion of the serial robot 400 which includes several links 401, which can be formed as a bent shape, and can transfer actuation from one link to another.

In some embodiments, by pushing or pulling actuation transfer component 402 or actuation transfer component 403 one engages and/or releases a release mechanism (RM) of a long serial mechanism (LSM). The actuation transfer component 402 or actuation transfer component 403 shown in FIG. 4 is optionally designed for linear actuation.

In some embodiments, by rotating the actuation transfer components 402 403, or shafts 402 403 one may activate a moving mechanism (MM) and thereby one or more engaged serial robot's link(s).

In some embodiments activating a moving mechanism (MM) and the serial robot's joints is optionally accomplished using two rotary actuators and a linear actuator.

An aspect of some embodiments of the invention relates to the following structure and actuation.

A first example structure, named herein type-A:

1. A mechanism (M) having multiple degrees of freedom connecting multiple links.

2. At least two long serial mechanisms (LSM).

3. Activating the LSM can be done in a forward direction (AFD) or in a backwards direction (ABD).

4. The two LSMs are optionally situated side by side in the structure.

5. Each one of the LSMs is a concatenation of a set of n sub-mechanisms.

6. Each one of the sub-mechanisms includes a backlash mechanism (BM), a moving mechanism (MM) and in some embodiments optionally also a release mechanism (RM).

7. Each link includes two side-by-side situated sub-mechanisms located on two side-by-side LSMs. In some embodiments one driving mechanism (DM) is optionally attached to each joint.

8. Consider two consecutive sub-mechanisms in a LSM and let MM1 MM2 be corresponding moving mechanisms of the two consecutive sub-mechanisms—if the configuration of their backlash mechanism (BM) dictates that activating MM1 will also activate MM2 the two consecutive sub-mechanisms are termed engaged.

In some embodiments engagement can be in one of the two options—forward engagement (FE), or backward engagement (BE).

A First Example Actuation:

9. Activating both LSMs enough times in one direction, for example ABD, optionally results in all their BMs being engaged, FE (forward engaged) or BE (backward engaged). In some embodiments activating both LSMs enough times in a ABD direction, for example, optionally results in all their BMs being engaged, FE or BE.

10. Following engagement of two consecutive sub-mechanisms (optionally as described in item 8 above), activating the first LSM, for example m₁ times, for example in AFD direction, optionally results in an engagement, for example FE, configuration of m₁ MMs beginning from a proximal sub-mechanism to a distal sub-mechanism.

11. Activating the second LSM m2<m1 times optionally results in an engaged configuration of m₂ MMs, optionally beginning from the proximal sub-mechanisms to the distal sub-mechanism.

12. Following acts described in items 8, 9, and 10 above:
a. m₂ of the first MMs of both LSMs are now in an engaged configuration;
b. Next m₂-m₁ MMs are in a configuration where one LSM is in an engaged configuration while another LSM is not engaged; and
c. The following MMs of both LSMs are not engaged.

13. A j^{th} degree of freedom of a mechanism M is optionally actuated as a result of the relative motion of two j^{th} MMs of the two j^{th} sub-mechanisms of the two LSMs as described in Table 1 below. The DM optionally drives a mechanism M's degree of freedom in a forward (F) direction or a backward (B) direction or two of the directions (BB, FF) or with zero motion (0).

14. In some embodiments the DM optionally actuates only if at least one of the MMs (through their BM) is engaged and actuated in the same direction. A FE+ABD optionally results in no movement of that MM. In some embodiments BE+AFD optionally results in no movement of that MM. In some embodiments when the LSM is not actuated in AFD nor in ABD optionally results in no movement of the MM. Such situations are termed non-actuated (NA). In some embodiments FE+AFD optionally results in a movement.

TABLE 1

| j^{th} First MM | j^{th} Second MM | Resultant j^{th} DM operation |
|---|---|---|
| NA | NA | 0 |
| NA | FE + AFD | F |
| NA | BE + ABD | B |
| FE + AFD | NA | B |
| FE + AFD | FE + AFD | 0 |
| FE + AFD | BE + ABD | BB (longer B movement) |
| BE + ABD | NA | F |
| BE + ABD | FE + AFD | FF (longer F movement) |
| BE + ABD | BE + ABD | 0 |

15. In some embodiments, some or all sub-mechanisms have a RM which detaches a corresponding DM from an MM.

16. In some embodiments, some or all joints have a RM which releases the MM from at least one LSM.

17. In some embodiments some or all RMs are optionally actuated together from a proximal end of the mechanism.

Some Embodiments can be Described as Follows:

A Second Example Structure Named Herein Type-B:

1. A mechanism (M) with multiple degrees of freedom connecting multiple links.

2. One long serial mechanism (LSM).

3. Activating the LSM is optionally done in a forward direction (AFD) or in a backwards direction (ABD).

4. In some embodiments the LSM is optionally a concatenation of a set of n links.

5. In some embodiments each of the links is optionally based on a backlash mechanism (BM), a driving mechanism (DM), a moving mechanism (MM) and a release mechanism (RM).

6. Consider two consecutive links. Let MM1 MM2 be their corresponding moving mechanisms. If the configuration of their BM is operated so that activating MM1 will also activate MM2, we say that MM1 and MM2 are engaged. In some embodiments, engagement is optionally in one of two options—forward engagement (FE), and backward engagement (BE).

A Second Example Actuation:

7. Activating the LSM enough times (for example in an ABD direction) optionally results in all its MMs being engaged (for example BE engagement).

8. Following (7), activating the LSM m1 times (for example in an AFD direction) optionally results in an engagement (for example FE) configuration of m1 MMs beginning from a proximal link toward a distal link. The following MMs of the LSM are optionally not engaged.

9. The DM optionally drives a mechanism M's degree of freedom in a forwards (F) or backwards (B) direction or with zero motion (0) when its corresponding MM is AFD, ABD or not actuated, respectively.

10. A sequence comprising from a first to a j^{th} degree of freedom of the mechanism M is optionally actuated as a result of the motion of the first to the j^{th} MMs.

11. It is noted that in some embodiments a FE+ABD will result with no movement of that MM, and similarly so BE+AFD and when the LSM is not actuated neither in an AFD nor in ABD. These situations are named non-actuated (NA). FE+AFD optionally result in a movement.

12. In some embodiments all links have a RM which detaches a corresponding DM from an MM.

13. In some embodiments, some or links have a RM which releases the MM from at least one LSM.

14. In some embodiments some or all RMs are optionally actuated together from a proximal end of the mechanism.

The mechanism described above as the first example structure may in some embodiments include: a mechanism M of multiple degrees of freedom connecting multiple links.

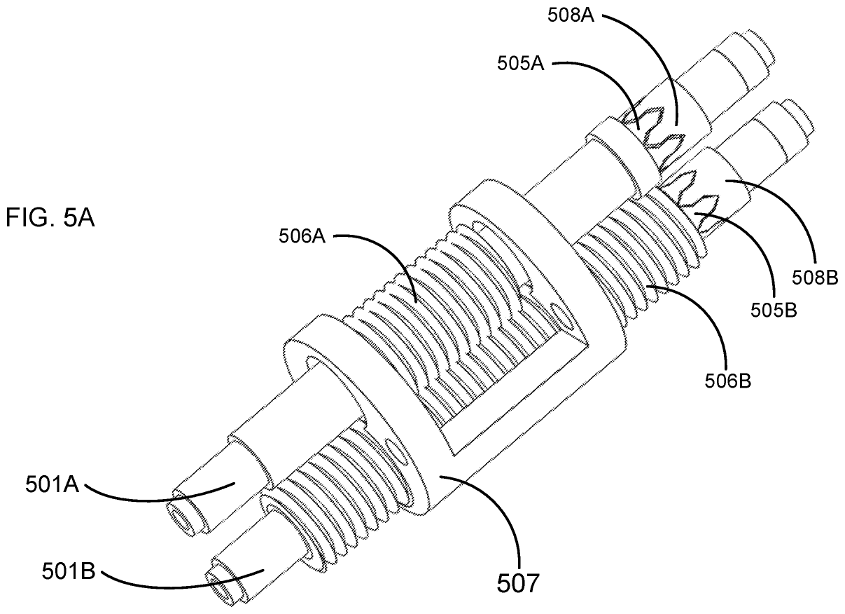

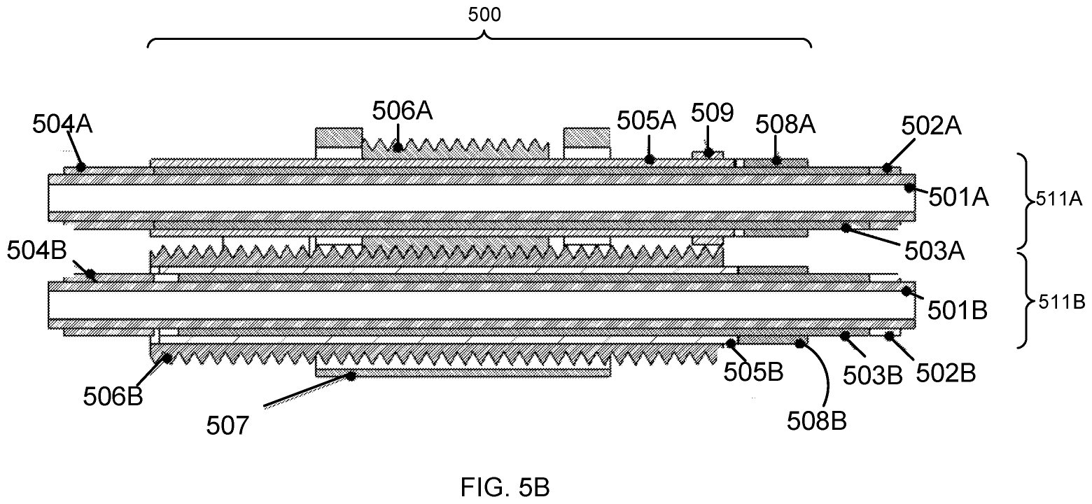

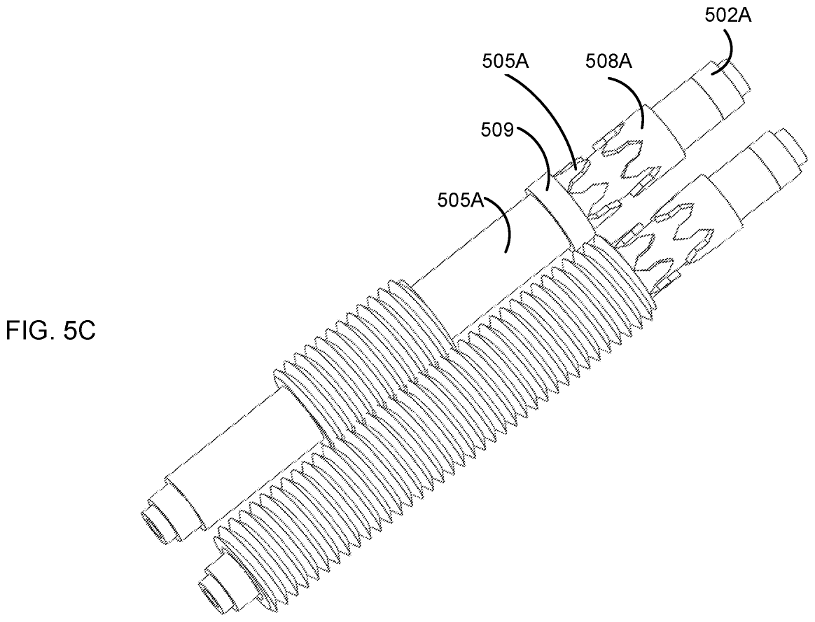

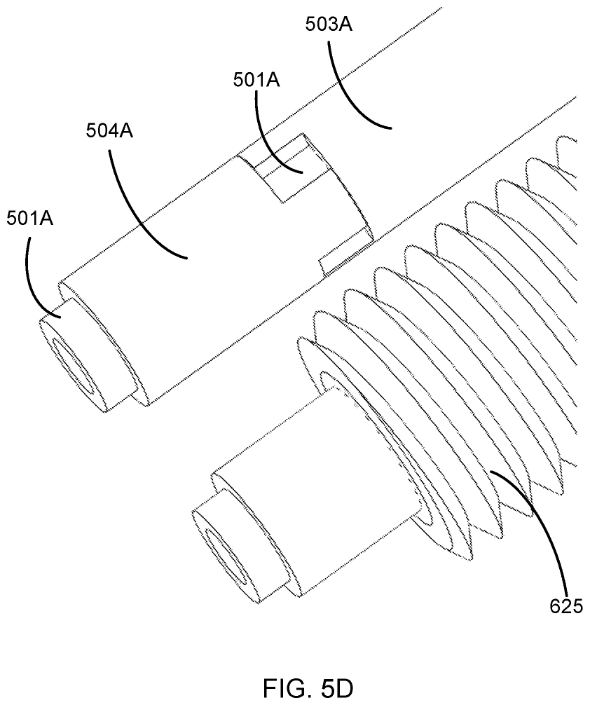

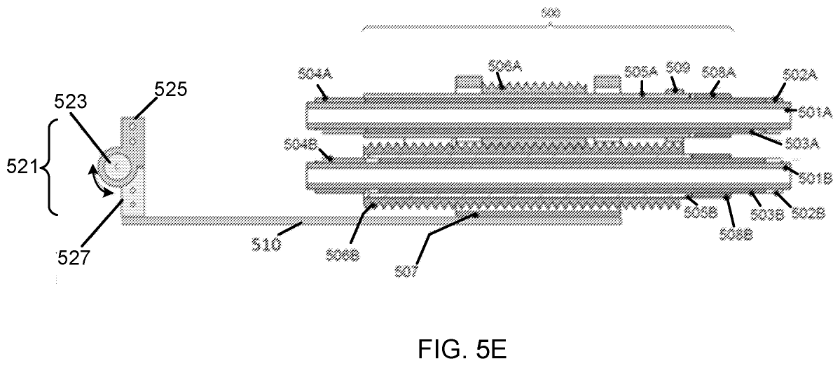

Reference is now additionally made to FIGS. 5A-5D, which are simplified illustrations of an example embodiment of the invention.

FIGS. 5A-5D shows an example embodiment of a link 500. FIGS. 5A, 5C and 5D are isometric views, and FIG. 5B is a cross sectional view, of the link 500.

The example embodiment of the link 500 includes:

shafts 501A and 501B;

mechanical stoppers 502A and 502B;

input parts of a backlash mechanism (BM) 504A and 504B;

output parts of a backlash mechanism (BM) 503A and 503B;

clutches, which act as release mechanisms (RMs), 505A and 505B and also 508A and 508B;

screws, acting as driving mechanisms (DMs) 506A and 506B;

a housing, acting as a moving mechanism (MM) 507; and

mechanical stopper 509.

It is noted that FIG. 5A-5C show an example embodiment of a type A mechanism, about which more is written later below.

In the example embodiments shown in FIGS. 5A-5C, references 501A and 501B are a rotating shaft type of LSM.

In the example embodiment of FIGS. 5A-5C:

the clutch 508A and the output part of the BM 503A are fixed to each other;

the clutch 505B and the screw 506B are fixed to each other; and

the screw 506A is fixed to the clutch 505A in a rotational direction, and is free to translate on a linear axis relative to the clutch 505A.

When the clutches 505A and 508A are interlaced, the clutch 505A will rotate when the output part of the BM 503A is rotated.

When the BM mechanisms (references 503A, 504A and 503B, 504B) rotate the screws 506A and 506B by rotating the shafts 505A and 505B. According to rotation of the screws 506A or 506B, the housing 507 is made to move according to a position of the screw 506A.

The above description applies, mutatis mutandis, for the clutches 505B and 508B when the clutches 505B and 508B are engaged.

FIG. 5C shows the clutch release mechanism, references 505A and 508A, disengaged.

In the example embodiment of FIG. 5C, references 502A and 509 are mechanical stoppers.

The mechanism shown in FIGS. 5A-5C optionally has two rotating shaft 501A and 501B types of LSMs 513A 513B, each of the LSMs 513A 513B optionally including multiple sub-mechanisms.

In some embodiments each of the links 500 in multiple links of the LSMs 513A 513B optionally includes such sub-mechanisms.

In some embodiments an LSM 513A 513B is a concatenation of long rods, or shafts 501A 501B, for example as shown in FIGS. 5A-5C.

In some embodiments the shafts 501A 501B are optionally connected by cardan joints.

In some embodiments the shafts 501A 501B are optionally constructed of HHS (Helical Hollow Strand) or HFS (Hollow Flexible shaft) and/or additional mechanisms as described below, so that rotating one of the shafts 501A 501B from its base results in rotating, in a “Roll” manner, all of the BMs which are.

In some embodiments AFD and ABD types of actuations optionally correspond to clockwise rotation and counterclockwise rotations.

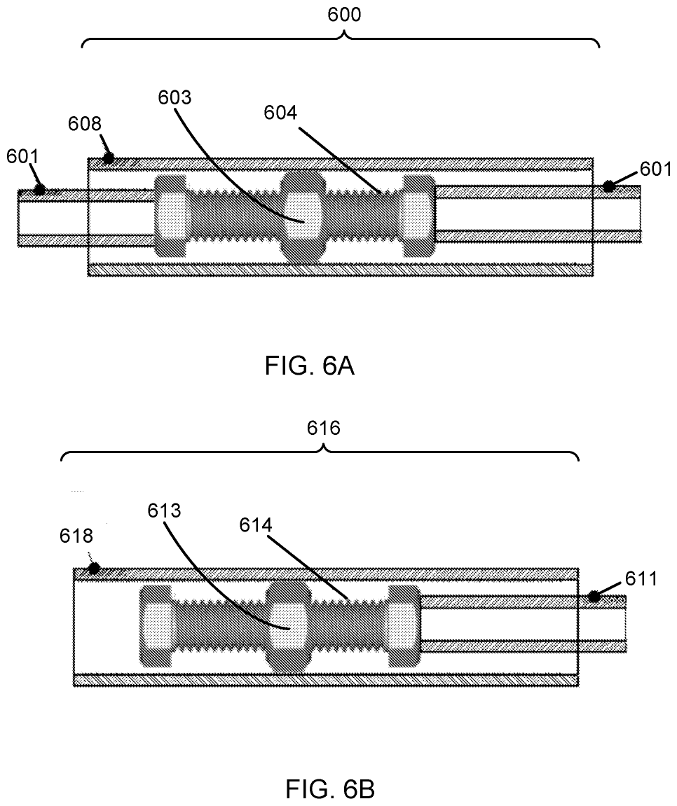

In some embodiments two shafts 501A 501B are optionally situated side by side in a link 500. A shaft in a single link 500 optionally includes a BM, which may optionally include a simple slot and screw structure, or as shown in FIGS. 6A, 6B, 6C, 7B and 9, further described below.

In some embodiments a loose-dog-clutch as depicted by the clutches 505A and 505B of FIGS. 5A-5C, is optionally fixed to the shaft 501A; and the BM 504B is optionally fixed to the shaft 501B of FIGS. 5A-5C.

In some embodiments when the BM 504B of shaft 501B, for example, is engaged, the BM 504B the BM 503B move together.

FIG. 5D shows an example embodiment of a backlash mechanism including:

the shaft 501A;

the output part 503A of the BM;

the input part 504A of the BM; and

the screw 506B.

In some embodiments a wire is optionally attached to two shafts at both of its ends. The wire is optionally taught when shafts are engaged and optionally loose otherwise.

In some embodiments an LSM includes a moving mechanism (MM) which may be embodied as a right or left threaded screw or bolt, coupled with a twin screw or bolt. Each link includes two screws: a leading screw—a long screw such as screw 506B, and a second screw such as screw 506A of FIGS. 5A and 5B, which can rotate and move while complying with the screw 506B. The screws 506A 506B are optionally followed by a release mechanism (RM). The RM is optionally a clutch mechanism, here embodied as a “dog clutch”, where coupling of two sides is achieved by engagement of toothed connections between two shafts as shown by clutch 508A of FIG. 5C.

In some embodiments leading-screws and moving-screws are concatenated on both LSMs, that is, there is no LSM that includes only leading-screws, and no LSM that includes only moving-screws. In such embodiments each link includes a leading screw and a moving screw. Such an embodiment is named a type-A embodiment herein.

In some embodiments all leading screws are on one LSM which is optionally called LSM-B, and all moving screws are on a second LSM, optionally called LSM-A.

The following description is relevant to both types of embodiments described in the two above paragraphs; nevertheless, for simplicity of description, the description is provided relating to the latter type of embodiment, the LSM-A and LSM-B design.

In an example embodiment, all backlash mechanisms from a base to an i-th link of the LSM-A are FE, that is, they all, for example, clockwise engaged; backlash mechanisms from the i-th link to a distal link are BE; backlash mechanisms from base to the j-th (where j<i) link of the LSM-B are FE as well; while backlash mechanisms of the LSM-B from the j-th link to the distal link are BE. Rotating both LSMs, for example from a proximal link in an AFD manner will result in clockwise rotation of both.

Note that the link from the base link to the j-th link will experience both their screws rotating clockwise, which results in zero, or small, linear movement of the moving screw A (B screw “tries to move A backwards”, but A “moves itself” forward). A list of such situations is given in Table 1.

Note also, that for the links beyond the j-th to the i-th link the leading screw B does not rotate when the moving screw rotate clockwise, which result in movement of all corresponding moving screws forward.

Note also, that the links beyond the i-th link remain without change. The links between the i-th link and the j-th link are differentiated, and a description is provided below how to take advantage of this and selectively activate specific links.

In such a manner one may choose which of the links to activate by adjusting a configuration of backlash mechanisms. To use a screw's movement a driving mechanism DM is optionally included. The driving mechanism may be embodied as a housing for the moving screw, such that the housing is confined to move linearly as the moving screw translates, optionally as shown by the housing 507 of FIGS. 5A and 5B.

It is noted that FIG. 5A-5D show an example embodiment of a type A mechanism, about which more is written later below.

Reference is now made to FIG. 5E, which is a simplified illustration of an example embodiment of the invention.

FIG. 5E shows an example embodiment of a link 500 as shown in FIGS. 5A-5D, connected to a hinge 521.

Similar reference numbers in FIG. 5E are used for components similar to reference numbers in FIGS. 5A-5D.

FIG. 5E shows, in addition to components shown in FIGS. 5A-5D, the housing 507 attached to a shaft 510. The shaft 510 is attached to a hinge 521, optionally a rotational hinge 521, optionally configured between two consecutive links.

In some embodiments a first part or leaf 527 of the hinge 521 is attached to the shaft 510, and a second part or leaf 525 of the hinge 521 is attached to a previous or next link (not shown). The hinge 521 can bend at an axis 523 of the hinge 521, potentially causing the previous or next link to form an angle relative to an axial direction of the link 500.

FIG. 5E show a configuration where the housing 507 of the link 500 can move and/or rotate the shaft 510, thereby moving or rotating the hinge 521.

In some embodiments an RM is optionally added, potentially enabling to obtain additional backlash mechanism configurations.

In some embodiments the RM is not included at all in the mechanism M.

For example, in order to obtain a state where links in a serial robot are described by: <FE, FE, FE, BE, *, *, . . . , *>, where a left component of the list in the brackets corresponds to a proximal end of a given LSM, one performs the following:

release all sub-mechanisms of that LSM by releasing, for example, the RM, for example the dog-clutch mechanism described herein;

perform ABD until all BMs are engaged; and

perform AFD three times.

In some embodiments one then re-engages all sub-mechanisms, that is, engages, for example, the dog-clutch mechanism described herein.

Reference is now additionally made to FIGS. 6A and 6B, which are simplified line drawing illustrations of two backlash mechanisms according to two example embodiments of the invention.

FIG. 6A shows an example embodiment of a backlash mechanism (BM) 600, including:

a first shaft 601;

a second shaft 608;

a nut 603; and

a bolt 604.

FIG. 6A shows a backlash mechanism (BM) where the first shaft 601 is a rotating shaft, and the second shaft 608 is a shaft that is optionally engaged with the first shaft 601 when the nut 603 reaches one of the ends of the bolt 604.

FIG. 6B shows an example embodiment of a backlash mechanism (BM) 616, including:

a first shaft 611;

a second shaft 618;

a nut 613; and

a bolt 614.

FIG. 6B shows a backlash mechanism (BM) where the first shaft 611 is a rotating shaft, and the second shaft 618 is a shaft that is optionally engaged with the first shaft 611 when the nut 613 reaches one of the ends of the bolt 614.

In some embodiments a BM is optionally constructed including a two-headed-bolt 604, with a nut 603 between the two heads of the bolt 604, optionally as depicted in FIGS. 6A and 6B, an actuation transfer component 601 that acts to rotate the BM with a resultant rotation in the driving mechanism 608.

FIG. 6B shows an example embodiment of a backlash mechanism (BM) 616, including:

an actuation transfer component 611, acting as a LSM;

a driving mechanism 618;

a nut 613 acting as a first part of a BM; and

a bolt 614 acting as a second part of a BM.

In some embodiments the BM 616 is optionally as depicted in FIG. 6B, where a rotating actuation transfer component 611 in an i^{th} link optionally results in no rotation of a next link's corresponding actuation transfer component until the nut 613 reaches up to one of the bolt's 614 heads.

Reference is now made to FIGS. 7A and 7B, which are simplified line drawing illustrations of two driving mechanisms according to two example embodiments of the invention.

FIG. 7A shows a spur gear 707, which optionally rotates and/or translates in accordance with rotations of worm screws 706A and 706B.

In some embodiments a movement, linear and/or rotational, of the spur gear 707, optionally constitutes a driving mechanism.

In some embodiments one or more of the worm screws 706A and/or 706B may optionally be moved linearly 702A 702B to engage with or dis-engage from the spur gear 707.

FIG. 7A shows two worm screws 706A 706B engaged with rolling shafts 709A 709B of LSMs. The spur gear 707 is optionally engaged with both worm screws 706A 706B. The spur gear 707 is free to translate linearly 708 in accordance to rolling directions of the worm screws 706A 706B. The movement may optionally be used to move a serial robot's link.

FIG. 7B shows an example embodiment of a type of backlash-and-activation. In such an embodiment movement of the LSMs is a rotation which may optionally be achieved by belts/tendons/etc. In the example embodiment of FIG. 7B the rotation is optionally achieved by in rotating pulley 703A and pulley 703B, and their rotation is passed over to a next link with belts/tendons, and so on. The pulley 704A and bevel gears (not shown), also known as a “sun gear”, or in some embodiments a friction wheel 701A are fixed together.

When rotating pulley 703A rotates, pulley 704A is caused to rotate, and rotate wheel 701A as well, when backlash between a pulley 703A and a pulley 704A is engaged. A pulley 703B, a pulley 704B and a wheel 701B will act in a same manner. A bevel gear 717, or in some embodiments a friction wheel, optionally acts as a planet pinion in a differential mechanism. The bevel gear 717 optionally translates in a same manner as the spur gear 707 of FIG. 7A.

In some embodiments the gear 717 operates as a moving mechanism (MM).

In some embodiments the pulleys 704A 704B operates as a backlash mechanism (BM).

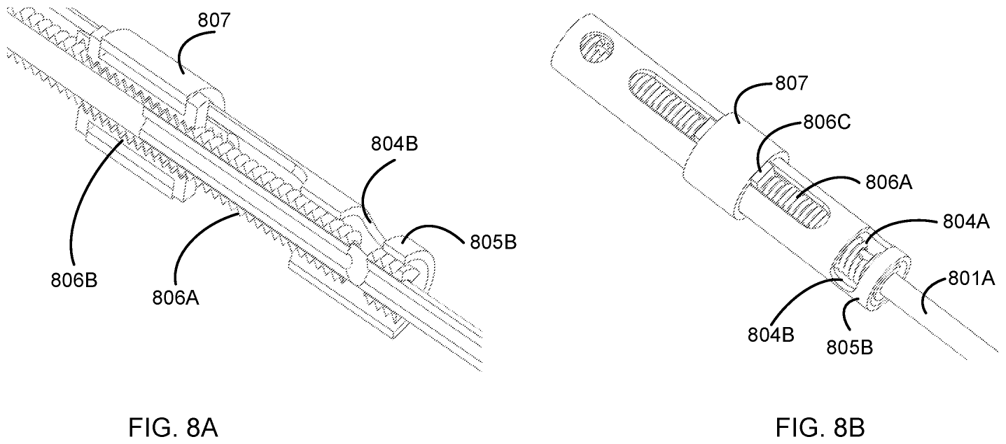

Reference is now made to FIGS. 8A and 8B, which are simplified line drawing illustrations of long serial mechanisms (LSMs) according to an example embodiment of the invention.

The example embodiment shown in FIGS. 8A and 8B is an example type A embodiment.

FIGS. 8A and 8B show an example embodiment where two LSMs are threaded one inside the other. The example of FIGS. 8A and 8B shows two LSMs combined into a single tube by threading the LSMs one into the other. The first LSM is the shaft 801A, and the second LSM is the tube 805B.

Rotating a shaft 801A will rotate a screw 806A when a backlash mechanism 804A is optionally engaged. Similarly when a backlash mechanism 804B is engaged, a moving screw 806B acts as a nut. In some embodiments the screw 806B optionally has a radial extension 806C which enables the backlash mechanism 804B to rotate the screw 806B while allowing a linear motion, which in turn moves a housing 807 which operates as a moving mechanism (MM).

Reference is now made to FIG. 9, which is a simplified line drawing illustration of two long serial mechanisms (LSMs) according to an example embodiment of the invention.

FIG. 9 depicts yet another embodiment where LSMs 901A 901B optionally move linearly rather than in a rotational manner. A backlash mechanism for the LSM 901A is optionally constructed as a mechanical linear stopper 903A 904A.

FIG. 9 also shows gears 906A 906B as rack gears, that is, linear gears, and a pinion gear 907. Driving the serial robot's joints is optionally done by harvesting the linear motion 902 of the pinion gear 907. In the embodiment shown by FIG. 9 if one moves the LSM-A 901A left and the LSM-B 901B right, only the third pinion gear 907 will move linearly 902.

A sequence of actions which can optionally reach such a state optionally includes:

(I) releasing all racks 903A 903B from the pinions 907, which can be performed with any release mechanism;

(II) moving the LSM-A 901A right until all BMs such as BM 903A 904A are engaged at the same direction, and then pushing the LSM-A 901A back to the left until engaging the BMs only at the two proximal links;

(III) moving the LSM-B 901B left, and then pulling the LSM-B 901B back to the right until engaging the BMs at the three proximal links;

(IV) engaging the racks to the pinions.

In some embodiments one would like a portion of the mechanism to move freely as a result of external forces, that is, to become “loose” in some portion of the serial robot.

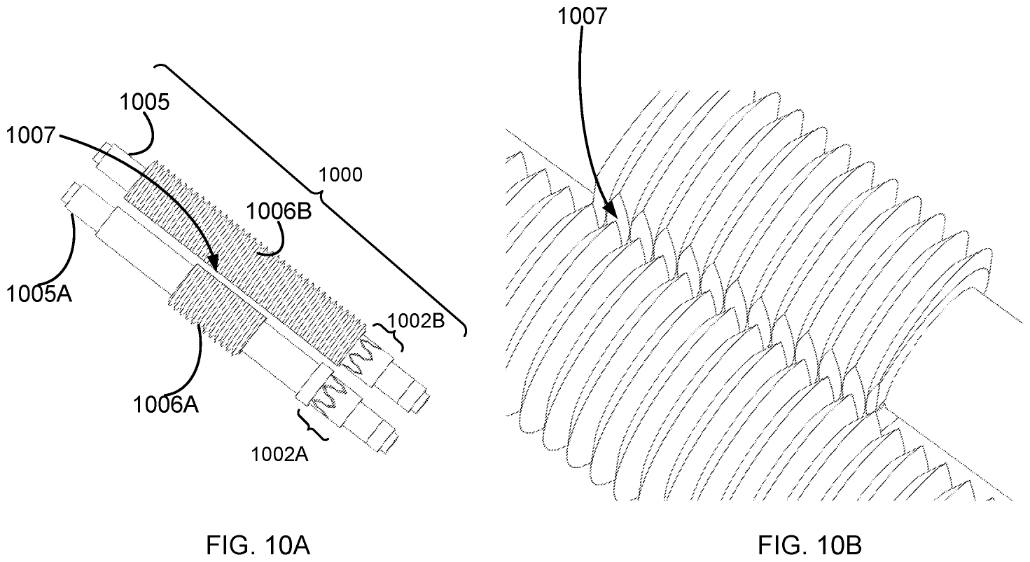

Reference is now made to FIGS. 10A and 10B, which are simplified line drawing illustrations of a link in a serial robot according to an example embodiment of the invention.

FIGS. 10A and 10B show an example embodiment which optionally enables a portion of the mechanism to be loose and move freely from external forces in some link 1000 of the serial robot.

FIGS. 10A and 10B show an example embodiment with screw threads 1006A 1006B of two shafts 1005A 1005B trimmed, cropped or flattened 1007 in a given angular sector of a circumference of the shafts 1005A 1005B. Rotating the shafts 1005A 1005B so the cropped screw sections face one another will result in a “loose”, dis-engaged link, while other portions of the shafts 1005A 1005B may optionally still be controllable.

Rotation of the shafts 1005A 1005B in more than one link potentially results in some links being engaged (screw threads 1006A 1006B are engaged) and some links being dis-engaged.

In some embodiments different links have different portions of an entire circumference of one or both of the screw threads 1006 1006B are cropped,

In some embodiments rotation of the shafts 1005A 1005B is designed to engage and dis-engage specific links, based on what portion of their screw threads 1006 1006B are cropped, and/or at what relative angle the screw threads 1006 1006B are cropped.

FIG. 10A also shows clutch mechanisms 1002A 1002B.

It is noted that the shafts 1005A 1005B may be rigid or flexible such as HHS.

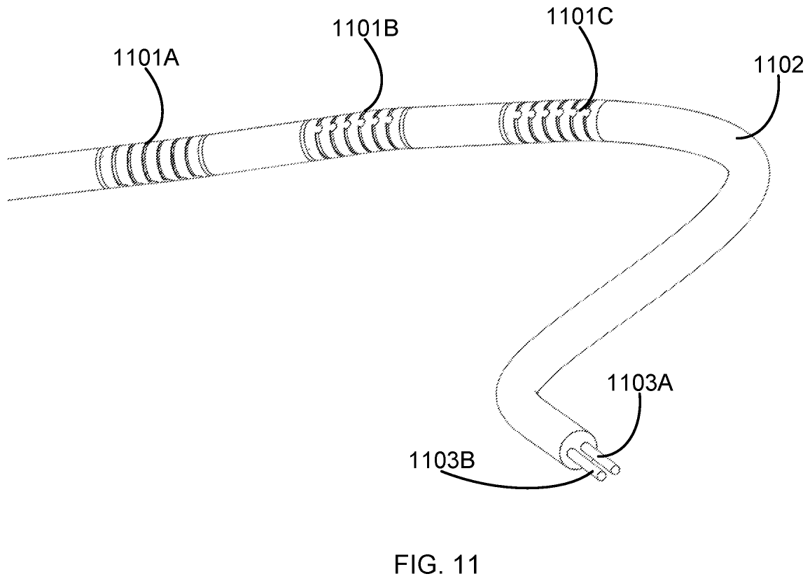

Reference is now made to FIG. 11, which is a simplified line drawing illustration of links of a serial robot according to an example embodiment of the invention.

FIG. 11 show a multi-link 1101A 1101B 1101C serial robot constructed as two LSMs 1103A 1103B.

FIG. 11 demonstrates a flexible shape of the serial robot.

In some embodiments the serial robot can be attached to a flexible guidance mechanism at its base.

It is noted that type A embodiments as described above can be understood with reference to type B embodiments by fixing (in terms of un-actuating) one of the LSMs.

A Type-B Embodiment is Described Below:

Note that one may also make do with just one LSM activated. To exemplify this let us consider an example embodiment as depicted in FIG. 4 or FIGS. 5A-5C. Taking the LSM-B (reference 403 in FIG. 4 and reference 501B in FIGS. 5A-5C, as fixed, and taking the RM of LSM-B as all engaged (or non-existent) while LSM-A is controlled, one can rotate the LSM-A and use the RM of the LSM-A when one wants to actuate only link i, one performs the following actions:

(I) Release LSM-A (via its release mechanism RM);

(II) Rotate LSM-A clockwise until all BM are engaged;

(III) Rotate the LSM-A counterclockwise i times;

(IV) Engage the RM of LSM-A;

(V) Rotate the LSM-A counterclockwise once (note that now all links from the proximal link to the i-th link are actuated).

In order to end up with only the i-th link actuated one needs to activate all links from the proximal link to the (i−1)-th link in a reverse direction.

(VI) Repeat actions (I)-(V) in a reverse direction for all links up to i−1.

A Flexible Base

In some embodiments the two types A and B described above are optionally implemented with a flexible tube which is optionally used as a catheter which has a robot at its distal end.

In some embodiments the system is optionally designed to include a proximal flexible portion. The distal, robotic end of the system is optionally fixed relative to a base segment or link of the serial robot. The fixing is optionally implemented in various ways.

In some embodiments, in an application where the serial robot is optionally positioned in a tunnel-like opening (e.g. aorta) which leads to a cavity to be manipulated, torus bellow is optionally inflated to expand against the tunnel walls. Such an embodiment may be used, for example to thread a serial robot to a heart, brain, sinuses or the gastric system.

Some examples of generalized embodiments include:

Generalized embodiment I: a design including a flexible tube sheath, a design including actuated joints, a design including a flexible tube sheath and actuated joints.

Generalized embodiment II: one or more legs of a parallel robot, also called a Stewart platform or a Gough-Stewart platform. A Stewart platform typically includes pairs of prismatic actuators, commonly hydraulic jacks or electric actuators, which can be embodiments of serial robots as described herein, attached in pairs to positions on the platform's baseplate, crossing over to three points on a top plate. Devices placed on the top plate can be moved in the six degrees of freedom in which it is possible for a freely-suspended body to move. The six degrees of freedom are the three linear movements x, y, z (lateral, longitudinal and vertical), and the three rotations pitch, roll, & yaw. The terms “six-axis” or “6-DoF” (Degrees of Freedom) platform are also used.

In some parallel robots a kinematic structure contains closed loops. In such a generalized embodiment the serial robot optionally contains a closed loop, where a first end of the serial robot is connected back to a second end of the same serial robot, in some cases via some other kinematic component. One can think of a parallel platform as a platform supported by several “legs”, or serial robots.

Generalized embodiment III: a serial robot according to embodiments described herein may optionally be used as part of an actuated flexible manifold.

In some embodiments of an actuated flexible manifold, several serial robots are optionally used as vertices defining a polygon. Several such polygons are optionally connected at their vertices, defining a multi-polygon surface.

A shape of the actuated flexible manifold is optionally controlled by controlling shapes and/or lengths and/or curves of one or more of the vertices, thereby optionally controlling the shape of the surface of the actuated flexible manifold.

Additional details about example embodiments of a Release Mechanism (RM) and a Backlash Mechanism (BM):

In some embodiments only one release mechanism is used, in one LSM, nevertheless providing all DOFs.

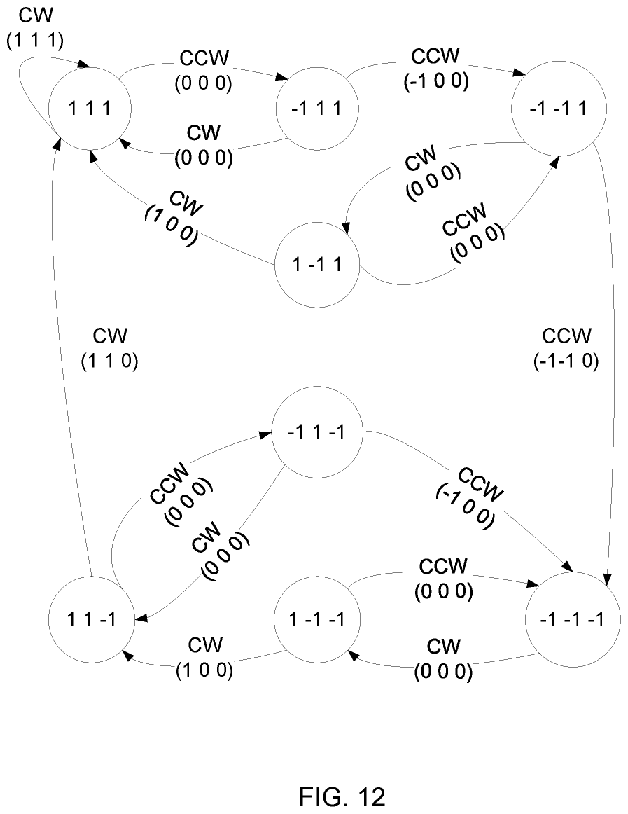

Reference is now additionally made to FIG. 12, which is a simplified line drawing illustration showing states and state transitions according to an example embodiment of the invention.

FIG. 12 shows a graph with nodes representing states and arrows representing transitions between states.

FIG. 12 shows states of an example embodiment of a serial vector according to an example embodiment of the invention. A state is shown as a circle containing notation of a vector with n values standing for backlash configurations of n links in the serial robot.

A backlash vector (a1 a2 a3) with all entries equal to −1 or 1, represents a backlash configuration where a1 is a proximal link's backlash configuration and a3 is a distal link's backlash configuration.

FIG. 12, by way of a non-limiting example, describes a backlash configuration space of a single LSM of a robot with, for example, 3 links. Each node, drawn as a circle, in the configuration space represents a backlash configuration. Arrows represent a backlash configuration change, for example performed by optionally rotating a shaft of an example embodiment shown in FIGS. 5A-5C.

FIG. 12 shows vectors, sets of three numbers within parentheses, corresponding to the angular velocities of 3 moving mechanisms, for example leading/moving screws, of three corresponding links. The vectors indicate possible paths for changing states in configuration space. A marking of “1” refers to a rotation which engages a BM.

In some embodiments a (1 1 1) backlash configuration either remains in the (1 1 1) backlash configuration, or moves to a (−1, 1, 1) backlash configuration.

In some embodiments a release mechanism is included. The release mechanism optionally enables to follow a path in the state space of the backlash configurations, and use the release mechanism to activate the LSM screws as desired, for example optionally going back to backlash configuration (1 1 1), optionally skipping a need to pass through undesirable backlash configurations which may change the resultant screws angular velocity.

By way of a non-limiting example, in some embodiments one may begin in a (1 1 1) backlash configuration, move to (−1 1 1) and to (−1 −1 1), use the backlash configuration to activate a proximal screw, and then directly move back to (1 1 1) without need for visiting the (1 −1 1) backlash configuration which may change the proximal MM angular velocity back to zero while returning to the (1 1 1) backlash configuration.

Two example embodiments for assembly of the backlash mechanisms described above include:

1. Where all backlash mechanisms are activated together (e.g. in the embodiment of FIGS. 5A-5C there is a shaft connected such that a roll rotation of the shaft rolls all backlash mechanisms at once). See, by way of some non-limiting examples, FIGS. 1A-1L and FIGS. 5A-5C.

2. A backlash mechanism i becomes “activated”, by way of a non-limiting example by a roll in the example embodiment of FIG. 4, only after an i−1 backlash mechanism is engaged. See, by way of some non-limiting examples, FIG. 6B and FIG. 9.

### Potential Implementations

An aspect of some embodiments of the invention includes improvements and enablement of surgery hereto difficult or impossible.

Some non-limiting examples include:

Improved laparoscopy, that is, surgical operation in a relatively open or vacant volume within a body;

Improved endoscopy, that is, operation within a body lumen, especially where a large number of degrees-of-freedom and a thin robot are to be used; and Operations on a brain and in spaces where a thin or narrow robot is important and the large number of degrees-of-freedom enables passing through narrow passageways such as between brain chambers.

Various embodiments of the invention have potential to enable NOTES surgeries. Some example applications of example embodiments include:

### Brain NOTES:

An example of current brain surgery includes an endoscopic trans-nasal approach.

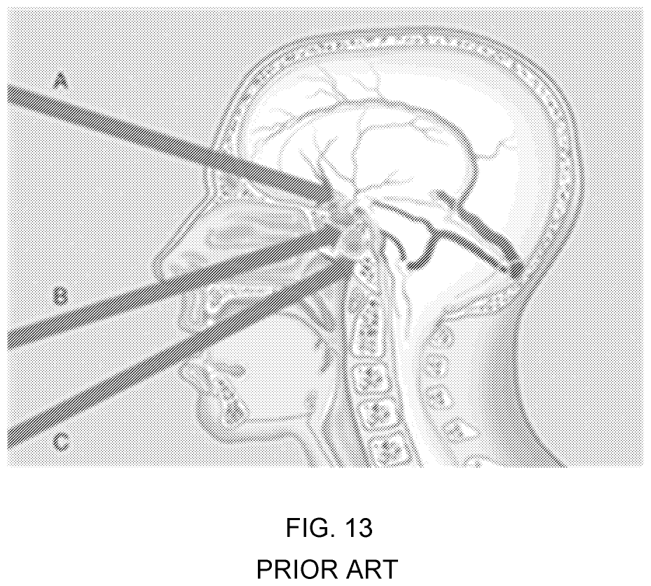

Reference is now made to FIG. 13, which is a simplified illustration of prior art NOTES approaches for brain surgery.

FIG. 13 shows an example of a trans-sphenoidal approach B, a sub-frontal trans-basal approach A, and a trans-oral approach C. it is noted that such NOTES approaches may suffer from long recovery periods.

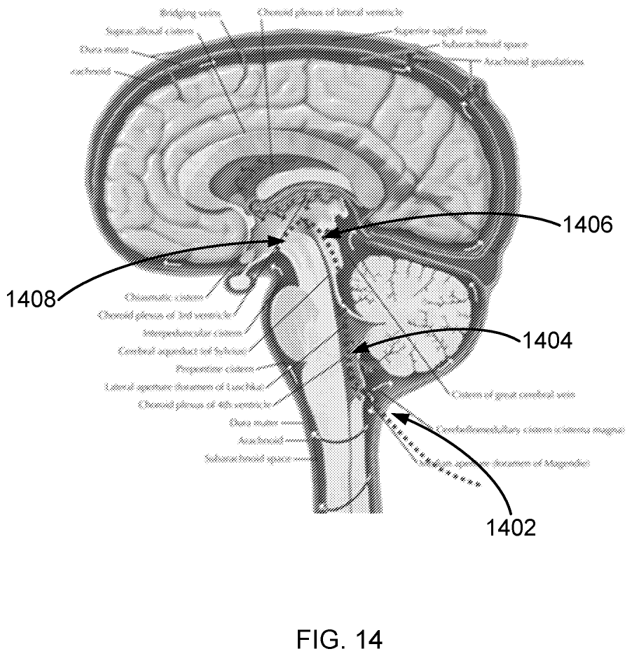

Reference is now made to FIG. 14, which is a simplified illustration of NOTES approaches for brain surgery according to an example embodiment of the invention.

Example NOTES approaches potentially using example embodiments of a serial robot as described herein can be performed via blood vessels, large blood vessels, or via the Median aperture.

In some embodiments of the invention, a serial robot is used to perform a NOTES brain surgery by approaching through a median aperture 1402, which is below the skull, and along narrow pathways into the skull. The narrow diameter of the serial robot potentially enable following a narrow path into the skull, optionally without drilling through the skull.

In some embodiments of the invention, a serial robot is used to perform a NOTES brain surgery by approaching through a median aperture 1402, through the choroid plexus of the 4^{th} ventricle 1404, through the cerebral aqueduct 1406, to the infundibular recess 1408.

A serial robot according to an example embodiment of the invention enables passing from ventricle to ventricle while maintain dexterity to control advance and movement of the serial robot.

It is noted that using a serial robot as described herein may also potentially enables using a trans-nasal or a trans-oral approach through smaller openings than used in prior art.

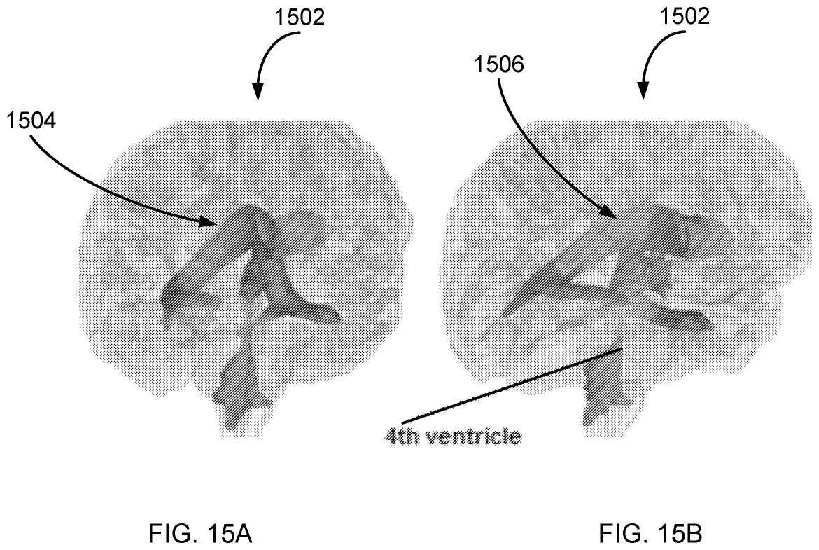

Reference is now made to FIGS. 15A and 15B, which are simplified illustrations of ventricles in the brain which an example embodiment of the invention potentially enables to perform NOTES surgery thereon.

FIGS. 15A and 15B are two different side views of the brain 1502, showing locations 1504 1506 of the ventricles.

A serial robot according to an example embodiment of the invention can be used for operating on deep brain tumors and/or lesion removal and/or for treatment of abnormal accumulation of CSF in ventricles of the brain.

It is noted that some orifices through which brain surgery is done are approximately 5-6 millimeters in diameter while an example embodiment of a serial robot such as shown, by way of some non-limiting examples, in FIGS. 1A-1N, can be fabricated with an outer diameter of 2.5 mm or lower.

A serial robot according to some example embodiments described herein can include just one or two LSMs, thereby maintaining a small diameter, even when the serial robot includes many links.

It is noted that some passageways through which brain surgery is done require a curvature radius as small as 3 centimeters or less, which is potentially achievable, by a serial robot such as shown, by way of some non-limiting examples, in FIGS. 1A-1N, which can provide a curvature radius of 10 mm or less.

A serial robot according to some example embodiments described herein can include angles between links of the serial robot, to form a curvature controlled by actuators at a proximal end of the serial robot. In some embodiments even a single actuator can be used to form several angles between several links, thereby forming a curve.

### Fetal NOTES Surgery

Fetal surgery is a surgical technique that is used to treat birth defects in fetuses in a uterus. Some example problems which are treatable by fetal intervention are usually anatomical problems for which correction in utero are feasible.

Open approach fetus surgery is currently used for: Prenatal repair of neural tube defects such as myelomeningocele, Hydrocephalus and Chiari malformation, Congenital diaphragmatic hernia, Congenital cystic adenomatoid malformation, Congenital heart disease, Pulmonary sequestration and Sacrococcygeal teratoma.

Minimally-invasive feto-scopic surgery has proven to be useful for some fetal conditions like twin-twin transfusion syndrome and Spina bifida and congenital diaphragmatic hernia.

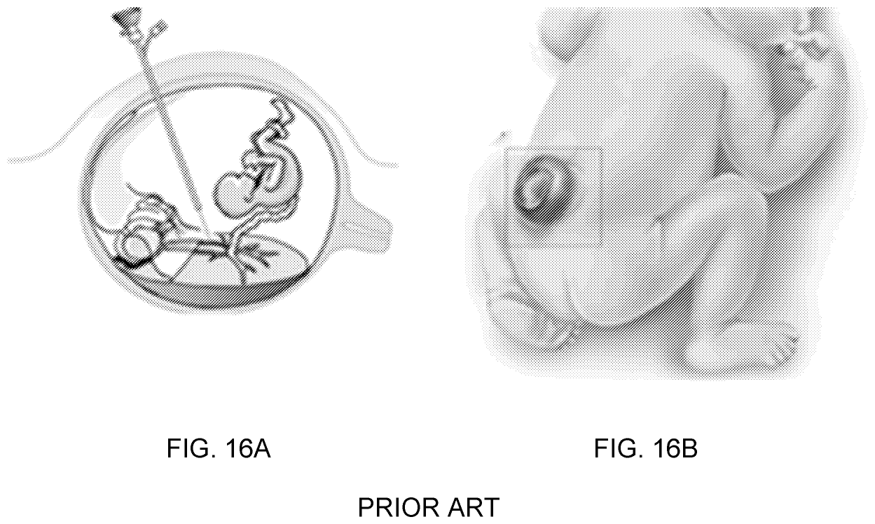

Reference is now made to FIGS. 16A and 16B, which are simplified illustrations of prior art fetal interventions.

FIG. 16A shows minimally-invasive feto-scopic surgery for a fetal condition such as Spina bifida.

FIG. 16B shows minimally-invasive feto-scopic surgery for a fetal condition such as Myelomeningocele.

Prior art endoscopic tools for such approaches have a limited dexterity and limit minimally-invasive feto-scopic surgery.

Embodiments of the invention described herein potentially extend the conditions under which the minimally invasive method can be used. A serial robot embodiments is optionally inserted through a small incision to access the uterus enabling improved dexterity within the uterus.

In some embodiments the serial robot is optionally used for a “double” minimal invasive surgery delivered to the fetus as well, by using the serial robot as well as other feto-scopic device(s).

### Cardiac Surgery

Trans-catheter Aortic Heart surgery is presently used for replacement of the aortic valve of a heart through blood vessels, for example TAVI (trans-catheter aortic valve implantation). The procedure is designed as a treatment for patients with aortic stenosis. Access is gained via the femoral artery. A delivery catheter with an aortic valve prosthesis is advanced to the narrowed valve. Using a balloon, delivered via a catheter inserted from a small incision, the diseased valve is pushed aside by inflation of a balloon. There are also therapies under development that seek to repair a mitral valve rather than replace it.

Some embodiments of the invention relate to a NOTES cardiac approach.

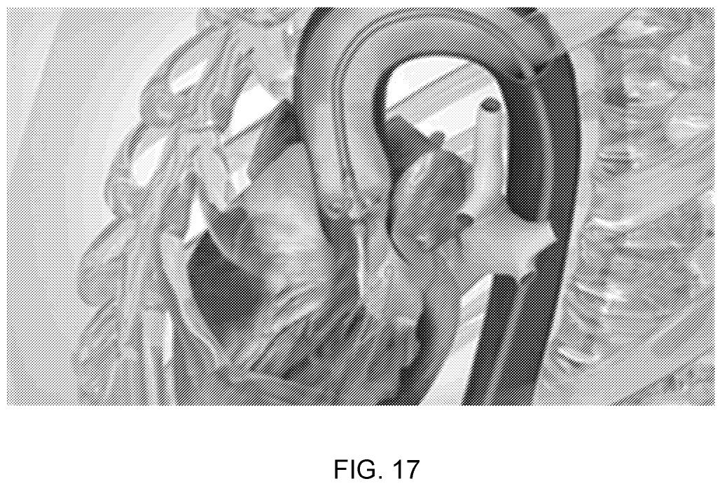

Reference is now made to FIG. 17 which is a simplified illustration of a NOTES cardiac approach according to an example embodiment of the invention.

A limited dexterity of a prior art catheter makes such a procedure cumbersome. Embodiments of the invention, by way of a non-limiting example the embodiments of FIGS. 1-11, are optionally used to access the heart's cavities in a trans-catheter aortic manner and maintain full dexterity and/or a large number of degrees of freedom for operating in the heart. For example, a diameter of the arteries through which the embodiments pass can range from 6 mm near an entry point to 30 mm near the heart, and the embodiments maintain full dexterity and/or a large number of degrees of freedom for operating at the heart.

In some embodiments, pathological conditions such as ventricular septal defect are optionally treated, or procedures that treat diseases affecting the tricuspid valve are performed.

Repair of the tricuspid valve is traditionally performed by open heart surgery and opening of the thoracic bone (sternotomy). Doctors connect the bone after the procedure to prevent movement and promote healing. The procedures for repairing the tricuspid valve may involve several types of repair, among others. Tricuspid valves that cannot open completely due to tricuspid valve stenosis can be repaired by surgery or a less invasive procedure called balloon valvuloplasty or valvotomy. These procedures are rarely performed. Balloon valvuloplasty is often used to treat infants and children with tricuspid valve stenosis. However, the valve tends to shrink again in operated adults; it is therefore generally reserved for adults who are too sick for surgery or who are waiting for valvular replacement.

Applying a serial robot as described herein potentially enables full dexterity within the heart, which potentially enables procedures such as ventricular septal defect or procedures that treat diseases affecting the tricuspid valve or similar to be performed via the aorta. Such a procedure can be performed by entering the common iliac artery advancing through the artery to the heart with a single robot. A second robot may be threaded via the external iliac vein and therefore enabling two fully dexterous robotic arm available for any cardiac procedure.

### Revascularization

Revascularization is presently usually done via laparotomy.

In some embodiments revascularization is optionally performed using embodiments of the invention.

### Trans-Colonic NOTES Surgery

In some embodiments Trans-colonic NOTES surgery is optionally performed using embodiments of the invention.

Trans-colonic NOTES surgery using embodiments of the invention are potentially an attractive option for treating colonic and abdominal diseases such as Trans-colonic endoscopic cholecystectomy.

Trans-Oral Robotic Surgery

Trans-Oral Surgery is a surgical technique for treatment of the mouth and throat via direct access through the mouth.

In some embodiments Trans-Oral Robotic Surgery is optionally performed using a serial robot implementation of the invention.

Such a serial robot is optionally used for more complex trans-oral procedures such as endoscopic submucosal dissection or even for entering the abdominal cavity.

In 2006 the American Society for Gastrointestinal Endoscopy and the Society of American Gastrointestinal and Endoscopic Surgeons organized a working group of surgeons and gastroenterologists to develop standards for the practice of this emerging technique. This group is known as the Natural Orifice Surgery Consortium for Assessment and Research (NOSCAR—www(dot)noscar(dot)org/resources/). According to a white paper on NOTES the group published some major areas of research should to be addressed before NOTES can become a viable clinical application for human patient: they name the following problems to be addressed: (1) Access to peritoneal cavity (2) Gastric (intestinal) closure (3) Prevention of infection (4) Development of suturing device (5) Development of anastomotic (non-suturing device) (6) Spatial orientation (7) Development of a multitasking platform to accomplish procedures (8) Control of intraperitoneal hemorrhage (9) Management of iatrogenic; intraperitoneal complications (10) Physiologic untoward events (11) Compression syndromes (12) Training other providers. As for (5) they state “For the simplest transgastric procedures (e.g., peritoneoscopy, specimen retrieval) a multitasking platform may be unnecessary. However, for NOTES to develop further, a multitasking platform is critical. Many important maneuvers for manipulating tissue are difficult to perform, even with a two-channel endoscope. For example, aggressive grasping of tissue to set up traction and counter-traction for exposure and division of structures is currently not possible. The flexibility of the endoscope, which provides a great advantage for traversing the gut lumen, is a disadvantage when applying force to tissue because it is very difficult to both push and pull at the same time. Fixation and stiffening the endoscope will be essential translumenal procedures. Because these procedures will require a team to manipulate instruments, devices with multiple ports are likely to be important. The role of robotics in this area seems promising, though a great deal of development work remains to be done. Voice activation technology may ultimately play a role in giving the therapist control of multiple devices, but initial development should focus on manual tools that ultimately can be modified for robotic control.”

In other words a robotic system for NOTES procedures should have sufficient dexterity (as opposed to tendon based mechanisms) deep within the body (as opposed robotic systems available which have a limited length), should demonstrate good repeatability (as opposed to catheter based procedures) and should be easily manipulated that is, it should have at least 6 DOF (typically catheters have 3 DOF). So the system enclosed herein may be a platform that conceptually addresses most technological problems (1, 4, 5, 6, 7 and 12) listed for NOTES.

Lastly note that available technologies for NOTES require a long preparation time, since the technique tolerance to mistakes is limited, when a full dexterity manipulator or even a redundant manipulator enables the surgeon much more flexibility while operating (like re-suturing when needed, handling bleeding, handling unexpected geometries etc.)

It is expected that during the life of a patent maturing from this application many relevant designs for driving mechanisms (DMs), long serial mechanisms (LSMs), backlash mechanisms (BMs), moving mechanisms (MMs) and release mechanisms (RMs) will be developed and the scope of the terms is intended to include all such new technologies a priori.

The terms “comprising”, “including”, “having” and their conjugates mean “including but not limited to”.

The term “consisting of” is intended to mean “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a unit” or “at least one unit” may include a plurality of units, including combinations thereof.

The words “example” and “exemplary” are used herein to mean “serving as an example, instance or illustration”. Any embodiment described as an “example or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-50. (canceled)

51. Apparatus, comprising a serial robot that comprises:
a chain of links, the chain having a distal end and a proximal end, and comprising at least:
a first link, comprising a first moving mechanism (MM),
a second link, coupled to the first link and distal from the first link, and comprising a second MM, and
a third link, coupled to the second link and distal from the second link;
an actuator, at the proximal end of the chain; and
a long serial mechanism (LSM) comprising:
a shaft, extending from the actuator distally along the chain, past the first link, the second link, and the third link, and
a series of driving mechanisms (DMs) distributed along the shaft, the series comprising at least a first DM and a second DM, disposed at the first link and the second link, respectively,
wherein:
the first link is configured such that actuation of the first MM acts upon the second link in a manner that adjusts a position of the second link with respect to the first link,
the second link is configured such that actuation of the second MM acts upon the third link in a manner that adjusts a position of the third link with respect to the second link,
the LSM is transitionable between:
an engaging state in which each DM of the series can potentially transfer rotation of the shaft into actuation of the MM of the respective link, and
a neutral state in which the first DM and the second DM cannot transfer rotation of the shaft into actuation of the MM of the respective link, and
the actuator is configured to, via only (i) transitioning the shaft between the neutral state and the engaging state, and (ii) rotation of the shaft, selectively:
independently of adjusting the position of the third link with respect to the second link, adjust the position of the second link with respect to the first link by causing the first DM to transfer rotation of the shaft into actuation of the first MM, and
independently of adjusting the position of the second link with respect to the first link, adjusting the position of the third link with respect to the second link by causing the second DM to transfer rotation of the shaft into actuation of the second MM.

52. The apparatus according to claim 51, wherein the LSM is an axially-innermost component in the serial robot.

53. The apparatus according to claim 51, wherein the LSM is an axially-innermost component in the serial robot.

54. The apparatus according to claim 51, wherein the LSM extends through each of the links in the chain.

55. The apparatus according to claim 51, wherein the LSM is flexible.

56. The apparatus according to claim 51, wherein the LSM is rigid.

57. The apparatus according to claim 51, wherein the shaft is tubular.

58. The apparatus according to claim 51, wherein the DMs of the series are axially fixed to the shaft.

59. The apparatus according to claim 51, wherein the DMs of the series are rotationally fixed to the shaft.

60. The apparatus according to claim 51, wherein:
the LSM is transitionable between the engaging state and the neutral state via axial movement of the LSM with respect to the chain, and
the actuator is configured to transition the LSM between the engaging state and the neutral state by axially moving the LSM with respect to the chain.

61. The apparatus according to claim 51, wherein the serial robot is configured such that rotating the shaft a full rotation while the LSM remains in the engaging state causes each DM of the series to transfer rotation of the shaft into actuation of the MM of the respective link.

62. The apparatus according to claim 51, wherein:
at the first link, the first DM and the first MM are arranged to define a first backlash mechanism (BM) arrangement that:
defines a first rotational driving-orientation of the shaft with respect to the first MM in which, while the LSM is in the engaging state, the first DM is in contact with the first MM such that any rotation of the shaft in a given direction is transferred by the first DM into actuation of the first MM, and
while the LSM is in the engaging state and the shaft is not in the first rotational-driving orientation, allows the shaft to be rotated in the given direction without actuating the first MM until the shaft reaches the first rotational orientation,
at the second link, the second DM and the second MM are arranged to define a second BM arrangement that:
defines a second rotational driving-orientation of the shaft with respect to the MM in which, while the LSM is in the engaging state, the second DM is in contact with the second MM such that any rotation of the shaft in the given direction is transferred by the second DM into actuation of the second MM, and
while the LSM is in the engaging state and the shaft is not in the second rotational-driving orientation, allows the shaft to be rotated in the given direction without actuating the second MM until the shaft reaches the second rotational orientation, and
the actuator is configured to:
adjust the position of the second link with respect to the first link independently of adjusting the position of the third link with respect to the second link by rotating the shaft in the given direction while (i) the LSM is in the engaging state, and (ii) the shaft is in the first rotational-driving orientation and not in the second rotational-driving orientation, and
adjust the position of the third link with respect to the second link independently of adjusting the position of the second link with respect to the first link by rotating the shaft in the given direction while (i) the LSM is in the engaging state, and (ii) the shaft is in the second rotational-driving orientation and not in the first rotational-driving orientation.

63. The apparatus according to claim 62, wherein the actuator is configured to continue to adjust the position of the second link with respect to the first link independently of adjusting the position of the third link with respect to the second link, even after the shaft has reached the second rotational driving-orientation, by:
transitioning the LSM into the neutral state,
rotating the shaft in the given direction past the second rotational driving-orientation while the LSM remains in the neutral state,
subsequently, returning the LSM to the engaging state, and
subsequently, continuing to rotate the shaft in the given direction.

64. The apparatus according to claim 63, wherein the actuator is configured to continue to adjust the position of the third link with respect to the second link independently of adjusting the position of the second link with respect to the first link, even after the shaft has reached the first rotational driving-orientation, by:
transitioning the LSM into the neutral state,
rotating the shaft in the given direction past the first rotational driving-orientation while the LSM remains in the neutral state,
subsequently, returning the LSM to the engaging state, and
subsequently, continuing to rotate the shaft in the given direction.

65. The apparatus according to claim 51, wherein:
at the first link, the first DM and the first MM are arranged to define a first backlash mechanism (BM) arrangement,
at the second link, the second DM and the second MM are arranged to define a second backlash mechanism (BM) arrangement,
the second BM arrangement differs from the first BM arrangement in a manner in which, starting at any given rotational orientation of the shaft, while the LSM remains in the engaging state:
rotation of the shaft in a given direction by a first amount of rotation engages the first DM with the first MM, and
rotation of the shaft in the given direction by a second, different amount of rotation engages the second DM with the second MM.

66. The apparatus according to claim 65, wherein:
even after rotation of the shaft by the second amount of rotation, the actuator is configured to continue to adjust the position of the second link with respect to the first link independently of adjusting the position of the third link with respect to the second link, by:
transitioning the LSM into the neutral state,
rotating the shaft in the given direction while the LSM remains in the neutral state such that the second DM skips past the second MM,
subsequently, returning the LSM to the engaging state, and
subsequently, continuing to rotate the shaft in the given direction.
